(19) United States
(12) Patent Application Publication
(10) Pub. No.: US 2025/0387360 A1
(43) Pub. Date: Dec. 25, 2025

(54) Methods and Compositions for Treatment of Addiction

(71) Applicant: University of Connecticut, Farmington, CT (US)

(72) Inventors: Yanjiao Zhou, Farmington, CT (US); Suresh Bokoliya, New Britain, CT (US)

(73) Assignee: University of Connecticut, Farmington, CT (US)

(21) Appl. No.: 19/242,833

(22) Filed: Jun. 18, 2025

Related U.S. Application Data
(60) Provisional application No. 63/661,629, filed on Jun. 19, 2024.

(51) Int. Cl. A61K 31/19; A61K 35/741; A61K 35/745; A61K 35/747; A61P 25/32

(52) U.S. Cl. CPC A61K 31/19; A61K 35/741; A61K 35/745; A61K 35/747; A61P 25/32

(57) ABSTRACT

Disclosed are agents, compositions, and methods including valeric acid, or a pharmaceutically acceptable salt thereof or a derivative thereof, for treating or preventing alcohol use disorder or alcohol risk consumption.

### RELATED APPLICATION

This application claims the benefit of priority to U.S. Provisional Application No. 63/661,629, filed on Jun. 19, 2024, the entire contents of which are incorporated herein by reference.

### GOVERNMENT SUPPORT

This invention was made with government support under AA027858 awarded by the National Institutes of Health. The government has certain rights in the invention.

### FIELD OF THE DISCLOSURE

The subject disclosure relates to microbiome metabolite-based agents/compounds, compositions and methods for treating or preventing alcohol use disorder in a subject. The subject disclosure particularly relates to the agents, compositions and methods including a short-chain-fatty-acid and their derivatives such as valeric acid and its derivatives for treating or preventing alcohol use disorder.

### BACKGROUND

Despite serious health and social consequences, effective intervention strategies for habitual alcohol binge drinking are lacking. Accumulating evidence in the past several years has established associations between the gut microbiome and microbial metabolites with drinking behavior, but druggable targets and their underlying mechanism of action are understudied. Therefore, development of novel therapeutic and preventative approaches is highly desirable.

### SUMMARY

In some aspects, the present invention provides a method for treating or preventing alcohol use disorder or alcohol risk consumption in a subject in need thereof, comprising administering to the subject an effective amount of valeric acid, or a pharmaceutically acceptable salt thereof.

In some aspects, the present invention provides a method of reducing alcohol consumption in a subject in need thereof, comprising administering to the subject an effective amount of valeric acid or a pharmaceutically acceptable salt thereof.

In some embodiments, the pharmaceutically acceptable salt is sodium valerate.

In some embodiments, the amount and/or frequency of alcohol consumption of the subject is reduced.

In some embodiments, the amount of alcohol consumption of the subject is reduced at least 10%, 30%, 50%, 70%, 90%, or 95%.

In some embodiments, the frequency of alcohol consumption of the subject is reduced to no more than once a week, once every two weeks, or once a month.

In some embodiments, the expression level of one or more genes comprising GPR56, KCNA10, PLN, H60C, IDI1, and/or SNORD34 is increased. In some embodiments, the expression level of one or more genes selected from the group consisting of GPR56, KCNA10, PLN, H60C, IDI1, and SNORD34 is increased. In some embodiments, the expression level of one or more genes selected from the group consisting of GPR56, KCNA10, PLN, H60C, IDI1, and SNORD34 is increased at least by 1.2, 1.5, 2, 2.5, 3, 3.5, 4, 5, 6, 7, 8, 9, or 10 times.

In some embodiments, the expression level of one or more genes comprising GPR158, RBM8A2, B4GALNT3, RASL10A, PTGS2, and/or PBK is decreased. In some embodiments, the expression level of one or more genes selected from the group consisting of GPR158, RBM8A2, B4GALNT3, RASL10A, PTGS2, and PBK is decreased. In some embodiments, the expression level of one or more genes selected from the group consisting of GPR158, RBM8A2, B4GALNT3, RASL10A, PTGS2, and PBK is decreased at least 10%, 30%, 50%, 70%, 90%, or 95%.

In some embodiments, the level of one or more inflammatory molecules in the subject is decreased. In some embodiments, the level of one or more inflammatory molecules in the subject is decreased at least 10%, 30%, 50%, 70%, 90%, or 95%.

In some embodiments, the one or more inflammatory molecules comprise prostaglandin-endoperoxide synthase 2 (PTGS2) and/or a mitogen-activated protein kinase (MAPK).

In some embodiments, the level of one or more bacteria of gut microbiome in the subject is increased. In some embodiments, the level of one or more bacteria of gut microbiome in the subject is increased at least by 1.2, 1.5, 2, 2.5, 3, 3.5, 4, 5, 6, 7, 8, 9, or 10 times.

In some embodiments, the one or more bacteria comprise Ileibacterium and/or Dubosiella.

In some embodiments, the level of gamma-aminobutyric acid (GABA) in the subject is increased. In some embodiments, the level of GABA in the subject is increased at least by 1.2, 1.5, 2, 2.5, 3, 3.5, 4, 5, 6, 7, 8, 9, or 10 times.

In some embodiments, the level of GAPA is increased in stool and/or amygdala of the subject.

In some embodiments, the level of anxiety of the subject is reduced.

In some embodiments, the valeric acid, or a pharmaceutically acceptable salt thereof, is administered orally.

In some embodiments, the method disclosed herein does not comprise administering carnitine or a derivative thereof to the subject.

In some aspects, the present invention provides a method of increasing the level of one or more bacteria of gut microbiomes in a subject, comprising administering to the subject an effective amount of valeric acid, or a pharmaceutically acceptable salt thereof, wherein the one or more bacteria comprise Ileibacterium and/or Dubosiella.

In some embodiments, the subject has alcohol use disorder.

In some embodiments, the amount and/or frequency of alcohol consumption of the subject is reduced.

In some embodiments, the amount of alcohol consumption of the subject is reduced at least 10%, 30%, 50%, 70%, 90%, or 95%.

In some embodiments, the frequency of alcohol consumption of the subject is reduced to no more than once a week, once every two weeks, or once a month.

In some embodiments, the expression level of one or more genes comprising GPR56, KCNA10, PLN, H60C, IDI1, and/or SNORD34 is increased. In some embodiments, the expression level of one or more genes selected from the group consisting of GPR56, KCNA10, PLN, H60C, IDI1, and SNORD34 is increased. In some embodiments, the expression level of one or more genes selected from the group consisting of GPR56, KCNA10, PLN, H60C, IDI1, and SNORD34 is increased at least by 1.2, 1.5, 2, 2.5, 3, 3.5, 4, 5, 6, 7, 8, 9, or 10 times.

In some embodiments, the expression level of one or more genes comprising GPR158, RBM8A2, B4GALNT3, RASL10A, PTGS2, and/or PBK is decreased. In some embodiments, the expression level of one or more genes selected from the group consisting of GPR158, RBM8A2, B4GALNT3, RASL10A, PTGS2, and PBK is decreased. In some embodiments, the expression level of one or more genes selected from the group consisting of GPR158, RBM8A2, B4GALNT3, RASL10A, PTGS2, and PBK is decreased at least 10%, 30%, 50%, 70%, 90%, or 95%.

In some embodiments, the level of one or more inflammatory molecules in the subject is decreased. In some embodiments, the level of one or more inflammatory molecules in the subject is decreased at least 10%, 30%, 50%, 70%, 90%, or 95%.

In some embodiments, the one or more inflammatory molecules comprise prostaglandin-endoperoxide synthase 2 (PTGS2) and/or a mitogen-activated protein kinase (MAPK).

In some embodiments, the level of one or more bacteria of gut microbiome in the subject is increased. In some embodiments, the level of one or more bacteria of gut microbiome in the subject is increased at least by 1.2, 1.5, 2, 2.5, 3, 3.5, 4, 5, 6, 7, 8, 9, or 10 times.

In some embodiments, the one or more bacteria comprise Ileibacterium and/or Dubosiella.

In some embodiments, the level of gamma-aminobutyric acid (GABA) in the subject is increased. In some embodiments, the level of GABA in the subject is increased at least by 1.2, 1.5, 2, 2.5, 3, 3.5, 4, 5, 6, 7, 8, 9, or 10 times.

In some embodiments, the level of GAPA is increased in stool and/or amygdala of the subject.

In some embodiments, the level of anxiety of the subject is reduced.

In some embodiments, the valeric acid, or a pharmaceutically acceptable salt thereof, is administered orally.

In some embodiments, the method disclosed herein does not comprise administering carnitine or a derivative thereof to the subject.

These and other aspects of the present invention are described in more detail below.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIGS. 1A-1D show effect of oral antibiotics on ethanol consumption, BEC and levels of SCFA in stool. FIG. 1A shows a schematic depiction illustrating the DID paradigm and Abx treatment. The light gray horizontal bar represents the 20% v/v ethanol drinking for two hours during the first three days and for four hours on the fourth day. The light orange horizontal bar symbolizes administration antibiotics (Abx) or PBS by oral gavage. The light blue horizontal bar represents the rest day post antibiotic treatment. The black arrow represents the measurement of ethanol consumption and BEC at the completion of the four hours ethanol drinking. FIG. 1B shows ethanol consumption normalized by body weight at end of four hours drinking period (n=14 mice/group). FIG. 1C shows BEC at end of four hours drinking period (n=14 mice/group). FIG. 1D shows effect of Abx administration on SCFA levels in stool (n=7 mice). In FIGS. 1B and 1D, a mixed ANOVA was used to compare two groups. The data in all three panels (FIGS. 1B-1D) are presented using box plots. The significant p-values are presented within the figure.

FIGS. 2A-2I show effect of sodium valerate on ethanol consumption, BEC, anxiety and diet. FIG. 2A shows this schematic illustrates incorporation of sodium valerate and NaCl supplementation into the DID paradigm. The light gray horizontal bar represent the 20% v/v ethanol drinking for two hours during the first three days and for four hours on the fourth day within the DID paradigm. The light green horizontal bar represents either sodium valerate in sodium valerate supplemented mice or NaCl supplementation in control mice. This supplementation continued throughout the entire experiment, except during DID sessions. The dashed black arrow indicates open field activity testing after completing 10 days of supplementation with sodium valerate or NaCl in cohort 1. Throughout this period, mice in the cohort 1 were also monitored for daily changes in body weight, food intake, and fluid intake. Additionally, the solid black arrow indicates the measurement of ethanol consumption and BEC through blood collection after the completion of the four hours ethanol drinking session in cohort 2. FIGS. 2B-2C show ethanol consumption (FIG. 2B), and BEC (FIG. 2C) in mice supplemented either with sodium valerate or NaCl (n=21 mice/group). FIGS. 2D-2F show percentage of time spent in the center (FIG. 2D), number of center entries (FIG. 2E), and distance travelled (FIG. 2F) in the center area during the open field test of mice supplemented with sodium valerate or NaCl (n=10 mice/group). In panel B-F, student t-test was employed to compare groups. In each of the four panels (FIGS. 2B-2F), the data are depicted using boxplots. FIGS. 2G-2I show daily body weight (FIG. 2G), food consumption (FIG. 2H) and intake of sodium valerate and NaCl (FIG. 2I) in respective groups over a 10-day period. Each line represents mean±SD for each group. The significant p-values are presented within the figure.

FIGS. 3A-3B show effect of sodium valerate supplementation on GABA levels. (FIG. 3A) The levels of GABA were measured in stool and amygdala tissues (n=7 mice/group), and (FIG. 3B) plasma samples (n=6 mice/group) of sodium valerate and NaCl control mice after 10 days of supplementation following the DID paradigm. The data are illustrated with boxplots. Student t-test was employed to compare groups. The significant p-values are provided within the figure.

FIG. 4 shows effect of sodium valerate supplementation on levels of histone acetylation. Acetylation of H3 and H4 in the amygdala of sodium valerate and NaCl control mice after 10 days of supplementation following the DID paradigm (n=7 mice/group). The data are illustrated with boxplots. Student t-test was employed to compare groups. The significant p-value is mentioned within the figure.

FIGS. 5A-5C show effect of sodium valerate on gene expression in the amygdala. FIG. 5A shows a PCA plot illustrating sample characteristics from both sodium valerate and NaCl groups (n=7 mice/group) based on gene expression levels, with each dot representing a sample. FIG. 5B shows heatmap displaying the top 50 upregulated and top 50 downregulated DEGs in mice supplemented with sodium valerate (left column) and NaCl (right column). The expression values are depicted as ranging from red (high expression) to pink (moderate), light blue (low), and dark blue (lowest expression). FIG. 5C shows highlights of some of the top canonical pathways identified by IPA that are affected by sodium valerate supplementation. The representation of pathway regulation is expressed as a percentage (top axis) using bar graphs, with respective p-values (bottom axis) derived from right-tailed Fisher's exact test represented by yellow line.

FIGS. 6A-6G show gut microbiome, gut-metabolites and gut-brain modules in sodium valerate supplemented mice. FIG. 6A shows stacked bar plot illustrating the percentage of top 25 genera present in stool samples from sodium valerate and NaCl supplemented mice (n=7 mice/group) as identified by sequencing of V4 16S amplicons. FIG. 6B shows PCA plots showing the variation in beta-diversity between mice supplemented with NaCl and sodium valerate at Day 0 and Day 10. Each dot represents a sample from an individual mouse. FIG. 6C shows displays of the changes of relative abundance of Ileibacterium after the 10-day period of sodium valerate and NaCl supplementation from the baseline day 0. FIG. 6D shows the differences in relative abundance of Ileibacterium between the groups at day 0 and day 10. FIG. 6E shows highlights of the variations in relative abundance of Ileibacterium within each group at day 0 and day 10. FIG. 6F shows demonstration of the differences in relative abundance of Dubosiella between the groups at day 0 and day 10. In FIGS. 6C-6G, the data are presented using box plots. FIG. 6G shows depiction of predicted gut-brain modules in mice supplemented with sodium valerate and NaCl. Relative abundance difference of specific taxa was identified using Wilcoxon-Sum Rank test. Here n=7 mice/group were used for gene expression analysis.

FIGS. 7A-7B show SCFA supplementation effect on ethanol consumption. FIG. 7A shows ethanol consumption and FIG. 7B shows BEC in mice supplemented with SCFAs and NaCl (n=7 mice/group). The data is depicted using boxplots. One way ANOVA was employed, followed by Tukey's post-hoc analysis to compare ethanol consumption and BEC levels between groups. The significant p-value is displayed within the figure.

FIG. 8 shows the effect of sodium valerate on alcohol drinking and blood ethanol levels were also tested in young female mice (n=7/group, 6-8 weeks old) using the same 4-day DID regimen. BECs level and ethanol consumptions were measured and compared between sodium valerate group and vehicle control group.

FIG. 9 shows dosage dependent effect of sodium valerate and BEC and alcohol intake. Different dosages of sodium valerate ranging from 20 mM to 200 mM were tested in male mice (n=7/group, 6-8 weeks old) using a 4-day DID regimen. Different dosages of sodium valerate were given through drinking water 30 min before the 2-hour drinking exposure at Day 1 and continued to day 4 except the drinking hours. 100 mM and 200 mM dosages can reduce drinking significantly compared to vehicle controls.

FIGS. 10A-10B show effects of sodium valerate on blood ethanol concentration (BEC) and ethanol consumption in a six-week Drinking in the Dark (DID) paradigm. Box plot of ethanol consumption (FIG. 10A) and blood ethanol concentration (BEC) (FIG. 10B) are shown over six weeks of a Drinking in the Dark (DID) paradigm in male mice (n=30). A two-way ANOVA with Tukey's post hoc test was used analysis and statistical significance is indicated as *p<0.05, **p<0.01.

FIGS. 11A-11D show effects of sodium valerate on excessive ethanol consumption in mice subjected to a 24-hours intermittent access, two-bottle choice drinking paradigm. Mean±SD values of ethanol consumption (FIG. 11A) and ethanol preference (FIG. 11B) are shown over 8 weeks of voluntary 20% ethanol intake in male mice (n=20). A significant escalation in ethanol intake was observed over time, with ****p<0.0001 compared to the first drinking session (analyzed using one-way repeated measures ANOVA followed by Tukey's post hoc test). After escalation, treatment with sodium valerate reduced both ethanol consumption (FIG. 11C) and preference (FIG. 11D) during the 4-week treatment phase. Statistical significance compared to baseline is indicated as **p<0.01, ***p<0.001, and comparisons to the NaCl control group are denoted as ## p<0.01 and ### p<0.001.

### DETAILED DESCRIPTION

The studies herein have identified a microbiome metabolite-based novel treatment using a short-chain-fatty-acid (e.g., valeric acid) and their derivatives (e.g., sodium valerate) that can reduce excessive alcohol drinking. Sodium valerate is a sodium salt of valeric acid-short-chain-fatty-acid with similar structure as γ-aminobutyric acid (GABA). It is demonstrated herein oral sodium valerate supplementation attenuates excessive alcohol drinking by 40%, reduces blood ethanol concentration by 53%, and improves anxiety-like or approach-avoidance behavior in male mice, without affecting overall food and water intake. Mechanistically, sodium valerate supplementation increases GABA levels across stool, blood, and amygdala. It also significantly increases H4 acetylation in the amygdala of mice. Transcriptomics analysis of the amygdala revealed that sodium valerate supplementation led to changes in gene expression associated with functional pathways including potassium voltage-gated channels, inflammation, glutamate degradation, L-DOPA degradation, and psychological behaviors. In addition, 16S microbiome profiling showed that sodium valerate supplementation shifts the gut microbiome composition and decreases microbiome-derived neuroactive compounds through GABA degradation in the gut microbiome. These findings indicate that the sodium valerate can serve as an innovative therapeutic avenue for the reduction of habitual binge drinking, potentially through multifaceted mechanisms.

Before the disclosed processes and materials are described, it is to be understood that the aspects described herein are not limited to specific embodiments, or examples, and as such can, of course, vary. It is also to be understood that the terminology used herein is for the purpose of describing particular aspects only and, unless specifically defined herein, is not intended to be limiting.

### Definitions

Compounds and materials are described using standard nomenclature. Unless defined otherwise, all technical and scientific terms used herein have the same meaning as is commonly understood by one of skill in the art to which this invention belongs.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

The terms “comprising”, “having”, “including”, and “containing” are to be construed as open-ended terms (i.e., meaning “including, but not limited to”) unless otherwise noted.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

As used herein, the term “administering” means the actual physical introduction of a composition into or onto (as appropriate) a subject, a host or cell. Any and all methods of introducing the composition into the subject, host or cell are contemplated according to the invention; the method is not dependent on any particular means of introduction and is not to be so construed. Means of introduction are well-known to those skilled in the art, and also are exemplified herein.

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the terms “treat,” “treating,” and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. These terms also encompass therapy and cure. Treatment means any way the symptoms of a pathological condition, disorder, or disease are ameliorated or otherwise beneficially altered. Preferably, the subject in need of such treatment is a mammal, preferably a human.

As used herein, “preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease). Prevention does not require that the disease or condition never occurs in the subject. Prevention includes delaying the onset or severity of the disease or condition.

As used herein, the term “effective amount” refers to the amount of a therapy, which is sufficient to reduce or ameliorate the severity and/or duration of a disorder or one or more symptoms thereof, inhibit or prevent the advancement of a disorder, cause regression of a disorder, inhibit or prevent the recurrence, development, onset or progression of one or more symptoms associated with a disorder, detect a disorder, or enhance or improve the prophylactic or therapeutic effect(s) of another therapy (e.g., prophylactic or therapeutic agent). An effective amount can require more than one dose.

Effective amounts may vary depending upon the biological effect desired in the individual, condition to be treated, and/or the specific characteristics of the composition according to the present invention and the individual. In this respect, any suitable dose of the composition can be administered to the patient (e.g., human), according to the type of disease to be treated. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the composition according to the present invention desirably comprises about 0.1 mg per kilogram (kg) of the body weight of the patient (mg/kg) to about 400 mg/kg (e.g., about 0.75 mg/kg, about 5 mg/kg, about 30 mg/kg, about 75 mg/kg, about 100 mg/kg, about 200 mg/kg, or about 300 mg/kg). In another embodiment, the dose of the composition according to the present invention comprises about 0.5 mg/kg to about 300 mg/kg (e.g., about 0.75 mg/kg, about 5 mg/kg, about 50 mg/kg, about 100 mg/kg, or about 200 mg/kg), about 10 mg/kg to about 200 mg/kg (e.g., about 25 mg/kg, about 75 mg/kg, or about 150 mg/kg), or about 50 mg/kg to about 100 mg/kg (e.g., about 60 mg/kg, about 70 mg/kg, or about 90 mg/kg).

The term “subject” or “patient” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject is a human, such as a human being treated or assessed for a disease, disorder or condition, a human at risk for a disease, disorder or condition, a human having a disease, disorder or condition, and/or human being treated for a disease, disorder or condition as described herein. In some embodiments, the subject does not suffer from an ongoing autoimmune disease. In one embodiment, the subject is about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age. Values and ranges intermediate to the above recited ranges are also intended to be part of this invention. In addition, ranges of values using a combination of any of the above-recited values as upper and/or lower limits are intended to be included.

As used herein, a “symptom” of a disease includes any clinical or laboratory manifestation associated with the disease, and is not limited to what a subject can feel or observe.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

### Composition and Uses Thereof

Excessive alcohol consumption poses a significant public health concern, leading to myriad of health and social problems as well as a substantial economic burden. Alcohol use disorder (AUD), which impacts nearly 29.5 million individuals with aged 12 and older in the US, frequently co-occurs with anxiety disorders. Drugs like Acamprosate, disulfiram, and naltrexone have received approval from the US FDA for treating AUD. Concurrently, several promising candidates, including ketamine, are presently under investigation. Nonetheless, the US FDA has not approved any new drugs for AUD treatment in the past two decades. Development of pharmacological interventions to reduce alcohol drinking remains a high priority for the mission of National Institute on Alcohol Abuse and Alcoholism. Binge drinking, excessive alcohol consumption in shorter period of time, is the most common and costly pattern of excessive alcohol use in the US. Studies indicate that one out of three young Europeans and North Americans regularly engage in binge drinking. Binge drinking has been linked to a heightened risk of developing AUD and neuropsychiatric disorders in later life.

Recent advances in the microbiome and alcohol research have shown that AUD negatively affects not only the liver and other organ systems but also the gastrointestinal system and its indigenous microbiota. Individuals with AUD exhibit alterations in their gut microbiome, characterized by reduced levels of bacteria associated with anti-inflammatory effects and the production of short-chain fatty acids (SCFAs)—the primary gut microbial metabolites derived from fermentation of non-digestible carbohydrates. Emerging evidence on binge drinking suggests that it leads significant alteration in microbiome, with some effects persisting from adolescence into adulthood of rats. These alterations may further accelerate the cycle of addiction via the gut-brain axis. Interestingly, the gut microbiome is closely linked to SCFAs production and alcohol consumption directly decreases the abundance of SCFA-producing bacteria, and lowers SCFA levels in stools and blood. SCFAs are vital in regulating immune responses and maintaining gut and blood-brain barrier integrity. SCFAs have also been shown to affect behavior, including those involved in reward, stress, and substance use disorders. In rats, administration of SCFAs, like sodium butyrate, reduced alcohol-induced liver damage and inflammation. Systemic use of sodium butyrate even produced antidepressant-like behavior in rats. In contrast, acetate, another SCFA, encourages ongoing heavy drinking. At the molecular level, modulation of epigenetic regulation and gene expression in the brain, potentially mediated by SCFAs, may contribute to their effects on alcohol drinking behavior.

GABA reported to play a crucial role in neuropsychiatric, neurological disorders, and AUD. Several studies reported lower GABA levels in abstinent individuals with AUD, particularly in the occipital cortex and anterior cingulate cortex. Inventor has discovered that given its structural similarities with GABA, valeric acid, a SCFA, can be a viable agent for its potential involvement in compensatory neurochemical adjustments linked to binge drinking.

There is a lack of progress in development of novel AUD treatment. Only one drug was approved in the past 14 years. The success of treatment using current therapies are limited. It is demonstrated herein that valeric acids target multiple aspects of AUD and comorbidity of AUD, thus can offer a more effective treatment.

Accordingly, in some aspects, the present invention provides a method for treating or preventing alcohol use disorder in a subject in need thereof, comprising administering to the subject an effective amount of valeric acid, or a pharmaceutically acceptable salt thereof.

In some aspects, the present invention provides a method of reducing alcohol consumption in a subject in need thereof, comprising administering to the subject an effective amount of valeric acid or a pharmaceutically acceptable salt thereof. The chemical structure of valeric acid is shown as follow:

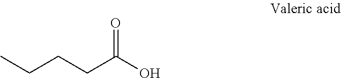

In some embodiments, the pharmaceutically acceptable salt is sodium valerate.

As used herein, “alcohol use disorder” or “AUD” refers to problem drinking that becomes severe. To be diagnosed with an AUD, individuals must meet certain criteria outlined in the Diagnostic and Statistical Manual of Mental Disorders (DSM). Under DSM-5, the current version of the DSM, anyone meeting any two of the 11 criteria during the same 12-month period receives a diagnosis of AUD. The severity of an AUD—mild, moderate, or severe—is based on the number of criteria met.

The invention may be used to treat alcohol risk consumption in a subject in need thereof. As used herein, and in accordance with the definition provided by National Institute on Alcohol Abuse and Alcoholism (NIAAA), “alcohol risk consumption” encompasses the following: (1) above moderate drinking; (2) exhibiting a pattern of binge drinking; and (3) heavy alcohol use. As used herein, “alcohol risk consumption” therefore refers to at least one of (1) to (3).

According to the “Dietary Guidelines for Americans 2015-2020,” (U.S. Department of Health and Human Services and U.S. Department of Agriculture), moderate drinking is up to 1 drink per day for women and up to 2 drinks per day for men.

According to the NIAAA, “binge drinking” is a pattern of drinking that brings blood alcohol concentration (BAC) levels to 0.08 g/dL. This typically occurs after 4 drinks for women and 5 drinks for men—in about 2 hours. Furthermore, the Substance Abuse and Mental Health Services Administration (SAMHSA), which conducts the annual National Survey on Drug Use and Health (NSDUH), defines binge drinking as 5 or more alcoholic drinks for males or 4 or more alcoholic drinks for females on the same occasion (i.e., at the same time or within 2 hours of each other) on at least 1 day in the past month. Accordingly, as used herein, “exhibiting a pattern of binge drinking” refers to a subject that is consuming alcohol in a manner than brings their blood alcohol concentration to 0.08 g/dL on at least one day in the past month.

SAMHSA also defines heavy alcohol use as binge drinking on 5 or more days in the past month. Accordingly, as used herein “heavy alcohol use” refers to a subject that has exhibited a pattern of binge drinking (as defined above) on 5 or more days in the past month.

In some embodiments, the amount and/or frequency of alcohol consumption of the subject is reduced.

In some embodiments, the amount of alcohol consumption of the subject is reduced at least 10%, 30%, 50%, 70%, 90%, or 95%.

In some embodiments, the frequency of alcohol consumption of the subject is reduced to no more than once a week, once every two weeks, or once a month.

The studies herein show that sodium valerate supplementation has a marked influence on gene expression in the amygdala. Accordingly, in some embodiments, the expression level of one or more genes comprising GPR56, KCNA10 (potassium voltage-gated channel, shaker-related subfamily, member 10), PLN (phospholamban), H60C (histocompatibility antigen 60c), IDI1 (isopentenyl-diphosphate delta isomerase, pseudogene 3), and/or SNORD34 (small nucleolar RNA, C/D box 34) (e.g., in an amygdala tissue of the subject) is increased. In some embodiments, the expression level of one or more genes selected from the group consisting of GPR56, KCNA10 (potassium voltage-gated channel, shaker-related subfamily, member 10), PLN (phospholamban), H60C (histocompatibility antigen 60c), IDI1 (isopentenyl-diphosphate delta isomerase, pseudogene 3), and SNORD34 (small nucleolar RNA, C/D box 34) (e.g., in an amygdala tissue of the subject) is increased. In some embodiments, the expression level of GPR56 is increased. In some embodiments, the expression level of KCNA10 is increased. In some embodiments, the expression level of PLN is increased. In some embodiments, the expression level of H60C is increased. In some embodiments, the expression level of IDI1 is increased. In some embodiments, the expression level of SNORD34 is increased.

In some embodiments, the expression level of one or more genes selected from the group consisting of GPR56, KCNA10, PLN, H60C, IDI1, and SNORD34 is increased at least by 1.2, 1.5, 2, 2.5, 3, 3.5, 4, 5, 6, 7, 8, 9, or 10 times.

In some embodiments, the expression level of one or more genes comprising GPR158, RBM8A2 (RNA binding motif protein 8A2), B4GALNT3 (beta-1,4-N-acetyl-galactosaminyl transferase 3), RASL10A (RAS Like Family 10 Member A), PTGS2 (prostaglandin-endoperoxide synthase 2), and/or PBK (PDZ binding kinase) (e.g., in an amygdala tissue of the subject) is decreased. In some embodiments, the expression level of one or more genes selected from the group consisting of GPR158, RBM8A2 (RNA binding motif protein 8A2), B4GALNT3 (beta-1,4-N-acetyl-galactosaminyl transferase 3), RASL10A (RAS Like Family 10 Member A), PTGS2 (prostaglandin-endoperoxide synthase 2), and PBK (PDZ binding kinase) (e.g., in an amygdala tissue of the subject) is decreased. In some embodiments, the expression level of GPR158 is decreased. In some embodiments, the expression level of RBM8A2 is decreased. In some embodiments, the expression level of B4GALNT3 is decreased. In some embodiments, the expression level of RASL10A is decreased. In some embodiments, the expression level of PTGS2 is decreased. In some embodiments, the expression level of PBK is decreased.

In some embodiments, the expression level of one or more genes selected from the group consisting of GPR158, RBM8A2, B4GALNT3, RASL10A, PTGS2, and PBK is decreased at least 10%, 30%, 50%, 70%, 90%, or 95%.

SCFAs may function in the brain through G protein-coupled receptors (GPCRs). Expression of GPR56 (or ADGRG1, adhesion G protein-coupled receptor G1) has been associated with antidepressant response. Conversely, GPR158 (G Protein-Coupled Receptor 158) is associated with depression after chronic stress. In some embodiments, the expression level of GPR56 is increased and the expression level of GPR158 is decreased in the subject (e.g., in an amygdala tissue or blood of the subject).

In some embodiments, the level of one or more inflammatory molecules in the subject is decreased. In some embodiments, the level of one or more inflammatory molecules in the subject is decreased at least 10%, 30%, 50%, 70%, 90%, or 95%.

PTGS2 is a prostanoids producer that responds to inflammation. Mitogen-activated protein kinases (MAPKs), a group of serine/threonine-specific protein kinases that regulate critical inflammatory processes. The main MAPK families in humans are the Extracellular Signal-Regulated Kinases (ERKs), c-Jun N-terminal kinases (JNKs), and p38 MAPKs. Downregulation of PTGS2 and MAPKs indicates anti-inflammatory effects. In some embodiments, the one or more inflammatory molecules comprise prostaglandin-endoperoxide synthase 2 (PTGS2) and/or a mitogen-activated protein kinase (MAPK). In some embodiments, the level of one or more extracellular signal-regulated kinases (ERK) (such as ERK1 and/or ER5) is decreased. In some embodiments, the level of one or more JNKs (e.g., JNK1, JNK2, and/or JNK3) is decreased. In some embodiments, the level of one or more p38s (e.g., p38-α (MAPK14), p38-β (MAPK11), p38-γ (MAPK12/ERK6), and/or p38-δ (MAPK13/SAPK4)) is decreased.

In some embodiments, the level of one or more bacteria of gut microbiome in the subject is increased. In some embodiments, the level of one or more bacteria of gut microbiome in the subject is increased at least by 1.2, 1.5, 2, 2.5, 3, 3.5, 4, 5, 6, 7, 8, 9, or 10 times.

In some embodiments, the one or more bacteria comprise Ileibacterium and/or Dubosiella.

In some embodiments, the level of gamma-aminobutyric acid (GABA) in the subject is increased. In some embodiments, the level of GABA in the subject is increased at least by 1.2, 1.5, 2, 2.5, 3, 3.5, 4, 5, 6, 7, 8, 9, or 10 times.

In some embodiments, the level of GAPA is increased in stool and/or amygdala of the subject. In some embodiments, the level of GAPA in stool of the subject is increased by at least by 1.2, 1.5, 2, 2.5, 3, 3.5, 4, 5, 6, 7, 8, 9, or 10 times. In some embodiments, the level of GAPA in stool of the subject is increased by at least by 1.2, 1.5, 2, 2.5, 3, 3.5, 4, 5, 6, 7, 8, 9, or 10 times.

In some embodiments, the level of anxiety of the subject is reduced.

In some embodiments, the valeric acid, or a pharmaceutically acceptable salt thereof, is administered orally.

In some embodiments, the method disclosed herein does not comprise administering carnitine or a derivative thereof to the subject.

In some aspects, the present invention provides a method of increasing the level of one or more bacteria of gut microbiomes in a subject, comprising administering to the subject an effective amount of valeric acid, or a pharmaceutically acceptable salt thereof, wherein the one or more bacteria comprise Ileibacterium and/or Dubosiella.

In some embodiments, the subject has alcohol use disorder.

In some embodiments, the amount and/or frequency of alcohol consumption of the subject is reduced.

In some embodiments, valeric acid, or a pharmaceutically acceptable salt thereof, is administered at least once per day. In some embodiments, valeric acid, or a pharmaceutically acceptable salt thereof, is administered at least 2 hours before drinking. In some embodiments, valeric acid, or a pharmaceutically acceptable salt thereof, is administered at least 1 hour before drinking. In some embodiments, valeric acid, or a pharmaceutically acceptable salt thereof, is administered at least 45 minutes before drinking. In some embodiments, valeric acid, or a pharmaceutically acceptable salt thereof, is administered at least 30 minutes before drinking. I an embodiment, valeric acid, or a pharmaceutically acceptable salt thereof, is administered at least 15 minutes before drinking. In some embodiments, valeric acid, or a pharmaceutically acceptable salt thereof, is administered at least 10 minutes before drinking. In some embodiments, valeric acid, or a pharmaceutically acceptable salt thereof, is administered at least 5 minutes before drinking. In some embodiments, valeric acid, or a pharmaceutically acceptable salt thereof, is administered at least twice per day. In some embodiments, valeric acid, or a pharmaceutically acceptable salt thereof, is administered at least thrice per day. In some embodiments, valeric acid, or a pharmaceutically acceptable salt thereof, is administered at least four times per day. In some embodiments, valeric acid, or a pharmaceutically acceptable salt thereof, is administered for a period of at least at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 3 week, at least 4 weeks, at least 5 weeks, at least 6 weeks, at least 7 weeks, at least 8 weeks, at least 9 weeks, or at least 10 weeks.

In some embodiments, valeric acid, or a pharmaceutically acceptable salt thereof, that can be used (therapeutic amount) is about 1 mM (millimolar) to about 1000 mM. In some embodiments, valeric acid, or a pharmaceutically acceptable salt thereof, that can be used is about 1 mM to 450 mM. In an embodiment, valeric acid, or a pharmaceutically acceptable salt thereof, that can be used is about 1 mM to 400 mM. In some embodiments, valeric acid, or a pharmaceutically acceptable salt thereof, that can be used is about 1 mM to 350 mM. In some embodiments, valeric acid, or a pharmaceutically acceptable salt thereof, that can be used is about 1 mM to 300 mM. In some embodiments, valeric acid, or a pharmaceutically acceptable salt thereof, that can be used is about 1 mg to 250 mM. In some embodiments, valeric acid, or a pharmaceutically acceptable salt thereof, that can be used is about 1 mM to 200 mM. In some embodiments, valeric acid, or a pharmaceutically acceptable salt thereof, that can be used is about 1 mM to 150 mM. In some embodiments, valeric acid, or a pharmaceutically acceptable salt thereof, that can be used is about 1 mM to 100 mM. In some embodiments, valeric acid, or a pharmaceutically acceptable salt thereof, that can be used is about 1 mM to 50 mM. In some embodiments, valeric acid, or a pharmaceutically acceptable salt thereof, that can be used is about 1 mg to 25 mg. In some embodiments, valeric acid, or a pharmaceutically acceptable salt thereof, that can be used is about 100 mM to 500 mM. In some embodiments, valeric acid, or a pharmaceutically acceptable salt thereof, that can be used is about 20 mM to 50 mM. In some embodiments, valeric acid, or a pharmaceutically acceptable salt thereof, that can be used is about 100 mM to 200 mM. In some embodiments, valeric acid, or a pharmaceutically acceptable salt thereof, that can be used is about 100 mM. In some embodiments, valeric acid, or a pharmaceutically acceptable salt thereof, that can be used is about 200 mM. In some embodiments, valeric acid, or a pharmaceutically acceptable salt thereof, that can be used is about 300 mM. In some embodiments, valeric acid, or a pharmaceutically acceptable salt thereof, that can be used is about 400 mM. In some embodiments, valeric acid, or a pharmaceutically acceptable salt thereof, that can be used is about 500 mM. In some embodiments, valeric acid, or a pharmaceutically acceptable salt thereof, that can be used is about 600 mM. In some embodiments, valeric acid, or a pharmaceutically acceptable salt thereof, that can be used is about 700 mM. In an embodiment, valeric acid, or a pharmaceutically acceptable salt thereof, that can be used is about 800 mM. In some embodiments, valeric acid, or a pharmaceutically acceptable salt thereof, that can be used is about 900 mM. In some embodiments, valeric acid, or a pharmaceutically acceptable salt thereof, that can be used is about 1000 mM.

In some embodiments, valeric acid, or a pharmaceutically acceptable salt thereof, that can be used (therapeutic amount) is about 400 mg/day to about 4000 mg/day, or can be about 400, 500, 600, 700, 800, 900, 1000, 1500, 2000, 2500, 3000, 3500, or about 4000 mg/day, or a combination of any of the foregoing values, or a range encompassing any of the foregoing values.

In one aspect, valeric acid, or a pharmaceutically acceptable salt thereof, can be administered once per day. In another aspect, valeric acid, or a pharmaceutically acceptable salt thereof, can be administered for at least 6 days up to 12 weeks. In one aspect, valeric acid, or a pharmaceutically acceptable salt thereof, are administered for 6, 7, 8, 9, or 10 days, or 2, 3, 4, 5, 6, 7, 8, 9, 10, 11, or 12 weeks, or a combination of any of the foregoing values, or a range encompassing any of the foregoing values. In another aspect, the composition can be self-administered. In any of these aspects, the subject can be concurrently consuming alcohol, or may not be concurrently consuming alcohol.

In one aspect, the composition comprises or the subject is administered one or more microbiome bacteria, for example, that improves valeric acid concentration in a subject, so as to treat or prevent alcohol use disorder or alcohol risk consumption in the subject, or reduce alcohol consumption in the subject. For example, In some embodiments, the one or more microbiome bacteria may comprise Lactobacillus, Bifidobacteria, or a combination thereof. In some embodiments, the bacteria is genetically engineered bacteria, wherein the genetically engineered bacteria provide valeric acid at a therapeutically effective amount (treatment concentration). In some embodiments, the bacteria is genetically engineered Lactobacillus. In some embodiments, the bacteria is genetically engineered Bifidobacteria. In some embodiments, the bacteria is genetically engineered Lactobacillus and Bifidobacteria.

In some aspect, disclosed is a method of treating or preventing alcohol use disorder or alcohol risk consumption in a subject, comprising administering to the subject a therapeutically effective amount of a composition that comprises one or more microbiome bacteria, for example, that improve valeric acid concentration in a subject. In some aspects, disclosed is a method of treating or preventing alcohol use disorder or alcohol risk consumption in a subject, comprising administering to the subject a therapeutically effective amount of a composition that comprises valeric acid, or a pharmaceutically acceptable salt thereof, and one or more microbiome bacteria that improve valeric acid concentration in a subject. The valeric acid, or a pharmaceutically acceptable salt thereof, may be administered in the same composition as, concurrently with or sequentially with the microbiome bacteria. In some embodiments, the one or more microbiome bacteria comprise Lactobacillus, Bifidobacteria, or a combination thereof. In some embodiments, the composition comprises genetically engineered bacteria, wherein the genetically engineered bacteria provide valeric acid at a therapeutically effective amount (treatment concentration). In some embodiments, the composition comprises genetically engineered Lactobacillus. In some embodiments, the composition comprises genetically engineered Bifidobacteria. In some embodiments, the composition comprises genetically engineered Lactobacillus and Bifidobacteria.

In some aspect, disclosed is a method of reducing alcohol consumption in a subject, comprising administering to the subject an effective amount of a composition that comprises one or more microbiome bacteria, for example, that improve valeric acid concentration in a subject. In some aspect, disclosed is a method of reducing alcohol consumption in a subject, comprising administering to the subject an effective amount of a composition that comprise valeric acid, or a pharmaceutically acceptable salt thereof, and one or more microbiome bacteria that improve valeric acid concentration in a subject. The valeric acid, or a pharmaceutically acceptable salt thereof, may be administered in the same composition as, concurrently with or sequentially with the microbiome bacteria. In some embodiments, the one or more microbiome bacteria comprise Lactobacillus, Bifidobacteria, or a combination thereof. In some embodiments, the composition comprises genetically engineered bacteria, wherein the genetically engineered bacteria provide valeric acid at a therapeutically effective amount (treatment concentration). In some embodiments, the composition comprises genetically engineered Lactobacillus. In some embodiments, the composition comprises genetically engineered Bifidobacteria. In some embodiments, the composition comprises genetically engineered Lactobacillus and Bifidobacteria.

### Pharmaceutical Compositions

In various aspects, the present disclosure relates to pharmaceutical compositions comprising a therapeutically effective amount of valeric acid or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In some embodiments, the disclosed pharmaceutical compositions comprise a therapeutically effective amount of valeric acid or a pharmaceutically acceptable salt thereof, and a pharmaceutically acceptable carrier, optionally one or more other therapeutic agents (e.g., other SCFAs), and optionally one or more adjuvants. The disclosed pharmaceutical compositions include those suitable for oral administration.

The pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the SCFAs the present disclosure can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for case of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration. This list of unit dosage forms is not intended to be limiting in any way, but merely to represent typical examples of oral unit dosage forms.

The pharmaceutical compositions disclosed herein can comprise valeric acid or a pharmaceutically acceptable salt thereof of the present disclosure as active ingredients, a pharmaceutically acceptable carrier, and optionally one or more additional therapeutic agents. The pharmaceutical compositions can be conveniently presented in unit dosage form and prepared by any of the methods well known in the art of pharmacy.

In one aspect, disclosed herein are oral dosage forms including the disclosed compositions and at least one pharmaceutically acceptable excipient. In another aspect, the pharmaceutically acceptable excipient can be a flavoring agent, a coloring agent, a preservative, a disintegrating agent, a coating, a bulking agent, a binder, thickener, a plasticizer, a carrier, or any combination thereof.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethyl-cellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

### EXAMPLES

### Example 1. Methods

### List of Abbreviations

Abx: antibiotic cocktail
BEC: blood ethanol concentration
DEG: differentially expressed genes
DeSeq2: differential expression analysis of RNA-Seq 2
DID: drinking in the dark
GABA: γ-aminobutyric acid
GBM: gut brain module
GPCR: G protein-coupled receptors
HDAC: histone deacetylase
IPA: ingenuity pathway analysis
NaCl: sodium chloride
MAPK: mitogen-activated protein kinases
PBS: phosphate-buffered saline
PCA: principal component analysis
SCFA: short-chain fatty acid

### Animals

Male C57BL/6J mice, (RRID: IMSR_JAX: 000664), aged 6-8 weeks, were obtained from Jackson Laboratories (ME, U.S.A) for the study. The mice were housed individually under a reversed 12-hours light/dark cycle, with lights off from 7:00 μm to 7:00 am. A minimum acclimation period of 2 weeks was provided for all animals before they were randomly assigned to their respective groups. Mice had free access to food (Irradiated chow, Teklad global 18% protein rodent diet, 2918, Inotiv, WI, U.S.A) and deionized water to acclimate to the testing environment. Body weight and daily food and fluid intake were monitored and noted for all animal experiments.

### Antibiotics Treatment

An antibiotic cocktail (Abx) comprised of 3.0 mg/mL vancomycin (Thermo Fisher Scientific, CA, USA), 6.0 mg/mL metronidazole (Sigma, MO, U.S.A), 6.0 mg/mL ampicillin (Sigma, MO, U.S.A), and 6.0 mg/mL neomycin (Janssen Pharmaceutical, Belgium) was given to mice via oral-gastric gavage for five consecutive days (250 μL Abx/gavage/mouse). In contrast, control mice were given phosphate-buffered saline (PBS; Thermo Fisher Scientific, CA, USA) as a control via oral-gastric gavage for five consecutive days, with each mouse receiving 250 μL of PBS per gavage.

### DID Paradigm for Binge-Like Ethanol Drinking and SCFA Administration

A standard 4-day drinking in the dark (DID) model was used to evaluate binge-like ethanol drinking patterns in mice. A 20% v/v ethanol solution was prepared by diluting 190-proof ethyl alcohol (Sigma, MO, U.S.A) with deionized water. During days 1-3, the water bottles were temporarily removed during the dark cycle of the mice, and the mice were provided with a tube containing the 20% v/v ethanol solution for a duration of two hours. On day four, the assessment of binge-like ethanol consumption took place, where the mice were given access to the 20% v/v ethanol solution for a period of four hours. The volume of fluid in the tube (read to the closest 100 μL) was measured immediately upon placement, and at two hours and four hours into the test. Following the completion of the four hours test, approximately 100 μL of blood was collected through cardiac puncture after CO₂ euthanasia. Blood samples were subjected to centrifugation at 10,000 rpm for 10 minutes. The determination of blood ethanol concentrations (BEC) was performed using an Analox AMI Analyzer (Analox Instruments, MA, U.S.A). The quantity of ethanol consumed was then calculated as grams per kilogram of the mouse's body weight.

All SCFA were used in sodium salt form. Briefly, sodium acetate (Sigma, MO, U.S.A), sodium butyrate (Sigma, MO, U.S.A), sodium valerate (Ambeed, IL, U.S.A), and sodium chloride (NaCl; Sigma, Mo, U.S.A) were prepared at 200 mM concentration freshly in drinking water every two days. These concentrations were chosen based on earlier studies conducted in mice [Qi Y., et al., Sodium acetate regulates milk fat synthesis through the activation of GPR41/GPR43 signaling pathway. Frontiers in nutrition 2023, 10:1098715; Daien C I, et al., Gut-derived acetate promotes B10 cells with anti-inflammatory effects. JCI insight 2021, 6 (7)]. All SCFA and NaCl solutions were pH verified to control for acidity effects on daily consumption. SCFAs were given to mice via oral drinking for 10 consecutive days before any experiment, and this regimen was maintained throughout the entire experiment.

### SCFA Measurement

The fecal samples (50-100 mg) were mixed with 1 mL of a 0.5% phosphoric acid solution (Sigma, MO, U.S.A). After collection, the samples were immediately frozen at −20° C. Upon thawing, the fecal content suspensions were thoroughly homogenized by vortexing for 2 minutes and then centrifuged at 18,000×g for 10 minutes. The resulting supernatant was extracted with ethyl acetate (Sigma, MO, U.S.A) and then centrifuged in glass tubes at 3,000×g for 10 minutes. The separated organic phase was analyzed using gas chromatography with a flame ionization detector (Shimadzu GC-QP2010 SE, Tokyo, Japan). Identification of SCFAs was achieved by comparison to the chemical standards (acetic acid, propionate acid, butyric acid, isovaleric acid, and valeric acid) (Sigma, MO, U.S.A).

### Open Field Activity Test

The open field activity test was conducted to assess the impact of sodium valerate supplementation on exploratory and anxiety-like behavior. This test is a widely used method for analyzing locomotor ability and anxiety-related emotional behaviors in C57BL/6J mice. The open field activity chamber, constructed from white Plexiglass and illuminated with daylight, had dimensions of 37.5 cm height×40 cm length×40 cm width. General locomotor activity was recorded for 10 min by an overhead camera and analyzed using an automated video tracking system (ANY-maze v.4.6, Stoelting, IL, U.S.A, RRID:SCR_014289). The primary variables of interest included the total distance traveled (cm), the number of entries in the center area (20 cm length×20 cm width), and the percentage of time spent in the center area of the open field.

### Measurement of GABA

GABA levels in plasma, stool, and amygdala samples were determined using an ELISA kit (LDN, Labor Diagnostika Nord, Nordhorn, Germany) following the manufacturer's instructions. Plasma samples were used directly without any additional preparation. For stool and amygdala, samples, they were first thawed and then homogenized in a solution consisting of 0.01N HCl (Thermo Fisher Scientific, MA, U.S.A), ImM EDTA (Thermo Fisher Scientific, MA, U.S.A), and 4 mM sodium metabisulfite (Thermo Fisher Scientific, MA, U.S.A). After homogenization, the samples underwent centrifugation at 3000×g for 5 minutes at 4° C. before GABA estimation. To summarize the assay procedure, plasma, homogenized stool, and amygdala samples, and the kit's standards were all processed on an extraction plate. They were subsequently derivatized using an equalizing reagent and subjected to a standard competitive ELISA conducted in GABA-coated 96-well microtiter strips. The optical density (OD) of the solution within the wells was rapidly read at 450 nm using a 96-well plate reader (iMark™ Microplate Absorbance Reader; Biorad, CA, U.S.A, RRID:SCR_023799). The OD data was employed to determine the GABA concentration via a standard curve.

### Measurement of Histone Acetylation in Amygdala

Amygdala samples were thawed, and the histone fractions were prepared using a commercial histone extraction kit (Abcam, Cambridge, UK) following manufacturers instruction. The bulk acetylation of histone H3 and H4 was determined using commercial ELISA kits according to the manufacturer's instructions (Abcam, Cambridge, UK). The OD of the samples was measured using a 96 well plate reader at 450 nm (iMark™ Microplate Absorbance Reader; Biorad, CA, USA). The amounts of acetyl histone H3 and H4 were measured by comparing to the kit supplied standards.

### RNASeq of Amygdala and Data Analysis

Total RNA was isolated from amygdala samples using the Direct-zol RNA Microprep Kit (Zymoresearch, CA, U.S.A). Quantification of total RNA and assessment of its purity ratios were performed using the NanoDrop 2000 spectrophotometer (Thermo Fisher Scientific, MA, U.S.A, RRID:SCR_018042).

Total RNA libraries were prepared for transcriptome sequencing using the Zymo-Seq RiboFree Stranded Total RNA library preparation kit (Zymo Research, CA, U.S.A) following the manufacturer's instructions. Further, rRNAs were depleted as part of the sample preparation process. Libraries were validated for length and adapter dimer removal using the Agilent TapeStation 4200 D1000 High Sensitivity assay (Agilent Technologies, CA, U.S.A, RRID:SCR_019398). Subsequent quantification and normalization were carried out using the dsDNA High Sensitivity Assay for Qubit 3.0 (Life Technologies, CA, U.S.A, RRID:SCR_020311). Sample libraries were then prepared for Illumina sequencing following the manufacturer's protocol (Illumina, CA, U.S.A). All the individual samples were consolidated into a single sequencing pool, with proportions targeting 30M reads/sample, and sequenced on the Illumina NovaSeq 6000 platform (Illumina, CA, U.S.A, RRID:SCR_016387).

Raw reads were subjected to adaptor removal and quality control including filtering of low-quality reads. Filtered reads were mapped to the mouse reference genome using STAR (Spliced Transcripts Alignment to a Reference, RRID:SCR_004463). Gene expression level was estimated by transcripts per million of transcript sequences. Principal component analysis (PCA) was performed to visualize overall gene expression differences between compared groups in several major principal components. The analysis of differential gene expression (DEG) was performed using the DESeq2 (RRID:SCR_015687) package for RNA-Seq data. The identified significantly regulated genes were displayed with varying adjusted p-value thresholds. The Benjamini-Hochberg procedure, also referred to as the false discovery rate (FDR) method, was applied to correct p-values for multiple testing. We utilized Ingenuity Pathway Analysis (IPA, RRID:SCR_008653) to predict pathways associated with differentially expressed genes and employed Gene Set Enrichment Analysis (GSEA, RRID:SCR_003199) to visualize the outcomes in a heatmap. Right tailed Fisher's exact test was performed in the IPA for identifying significant canonical pathways.

16S rRNA Sequencing and Microbiome Data Analysis

Microbial genomic DNA extraction from mouse stool samples was carried out using the Quick-DNA Fecal/Soil Microbe 96 Kit (Zymo Research, CA, U.S.A) following the manufacturer's protocols. The hypervariable region V4 of the bacterial 16S rRNA gene, 515F (5′-GTGYCAGCMGCCGCGGTAA-3′ (SEQ ID NO: 1)) and 806R (5′-GGACTACNVGGGTWTCTAAT-3′ (SEQ ID NO: 2)) was amplified and sequenced on the Illumina MiSeq platform (2×250 bp) (Illumina, CA, U.S.A). The raw sequencing reads were processed via the DADA2 V1.16 (RRID:SCR_023519) data processing pipeline to generate amplicon sequence variants (ASVs). Taxonomic assignments were made using the Silva database V138.1 (RRID:SCR_006423), with a classification confidence threshold set at p<0.5 for assigning unclassified taxa at their respective taxonomical levels. PCR negative controls and extraction controls for DNA extraction and sequencing were included in the analysis. Notably, all negative controls yielded fewer than 500 reads, indicating that background noise minimally impacted the data analysis. Sequencing reads from all the samples were rarified to 10,000 reads per sample. Further, ASVs were used for functional inference using PICRUSt2. The resultant Kyoto Encyclopedia of Genes and Genomes Orthologs (KOs) were used for gut brain modules (GBM) detection. GBM analysis was performed using the R version of the Gomixer tool and a non-parametric Wilcoxon test was used to compared NaCl and sodium valerate groups as GBM measures were not normally distributed. All of the microbiome analysis was done in R.

### General Statistical Approaches

General statistical analysis (non-microbiome and non-RNAseq analysis) were conducted using GraphPad Prism 8 (GraphPad, CA, USA, RRID:SCR_002798). For the calculation of ethanol consumption during the DID procedure, it was expressed as grams of ethanol per kilogram of body weight (g/kg body weight), where 20% ethanol intake was determined as follows: multiplying the drinking volume by the ethanol percentage within that volume and the density of ethanol, and then dividing this by the mouse's body weight in kilograms. Normality of data distributions was tested by Shapiro-Wilk test and by Q-Q plots. Levene's test was used to test equality of variances across groups. Unpaired Student's t-tests were used to compare behavioral testing, GABA levels, and histone acetylation measurements between the sodium valerate and NaCl groups. To evaluate the impact of antibiotic cocktail administration on ethanol consumption and BEC between the groups receiving Abx and PBS at baseline and after treatment, a mixed ANOVA test was employed, given repeated measures from the same mouse from the two time points. Similarly, mixed ANOVA tests were applied to compare various SCFA levels before and after Abx administration. Analysis of variance (ANOVA) was used for comparisons involving more than two groups when there are equal variances across samples. For instance, ordinary one-way ANOVA was employed to assess the impact of all SCFA supplementation on ethanol consumption and BEC. Corrections for multiple comparisons in ANOVA were made using Tukey's post-hoc test.

The food intake was assessed by providing a pre-weighed amount of food in their cage hopper and then weighing the remaining food inside the cage and in the hopper daily. To assess daily fluid intake, the sodium valerate and NaCl bottles were pre-weighed, and the remaining amount was deducted after the measurement period. Food and fluid intakes were normalized to mouse body weight by dividing the average intake by the average body weight.

For all above analysis, the p-values were reported with the respective F- or T-statistic and associated degrees of freedom (df).

### Example 2. Results

### Antibiotic Administration Increases Ethanol Consumption but Reduces Fecal SCFA Levels

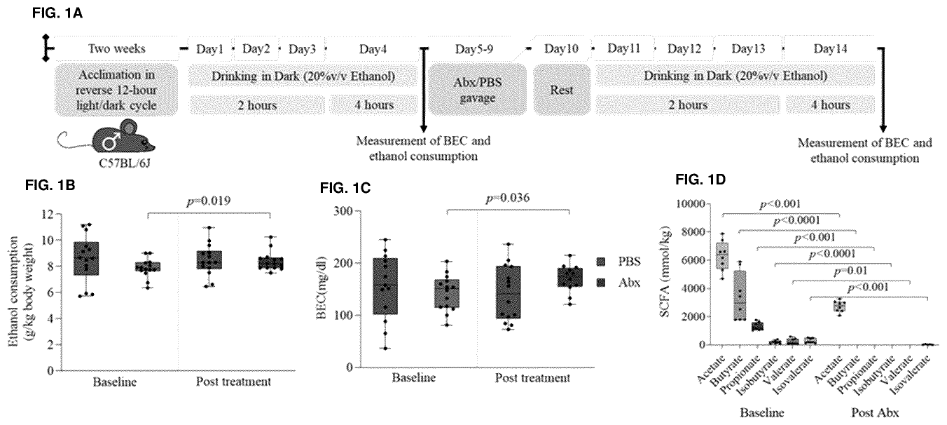

Ethanol consumption (unit: g/kg body weight) and BEC (unit: mg/dl) was measured in adult male mice (n=14 mice/group) using a DID paradigm at baseline and after Abx treatment. A schematic diagram illustrating the administration of Abx and the DID procedure is depicted in FIG. 1A. At baseline, no statistically significant differences in ethanol consumption (FIG. 1B) or BEC were noted (FIG. 1C). Following a 10-day treatment period with Abx, we once again employed the DID paradigm in both the Abx-treated mice and PBS control mice. Notably, after this treatment period, the Abx-treated mice exhibited a significantly higher level (p=0.03, t=2.44, df=11; FIG. 1B) of ethanol consumption and BEC (p=0.03, t=2.47, df=11; FIG. 1C) compared to their baseline levels. In contrast, the PBS control mice did not display a significant difference in either consumption or BEC when compared to their baseline levels.

To test whether the levels of SCFAs are altered in Abx treated mice, the SCFA at baseline and after Abx treatment was analyzed in the mice stool. At baseline, SCFAs such as acetate, butyrate, and propionate are usually found at high concentrations, while isobutyrate, valerate, and isovalerate are found at lower concentrations (n=7 mice). All SCFAs were significantly abolished after antibiotic treatment (p<0.05), with only minimal levels of acetate (mean±SD=2722.21±372.26 mmol/kg stool) and isovalerate (mean±SD=23.8±17.02 mmol/kg stool) remaining (FIG. 1D).

### Sodium Valerate Supplementation Reduces Ethanol Consumption, BECs, and Anxiogenic Behavior

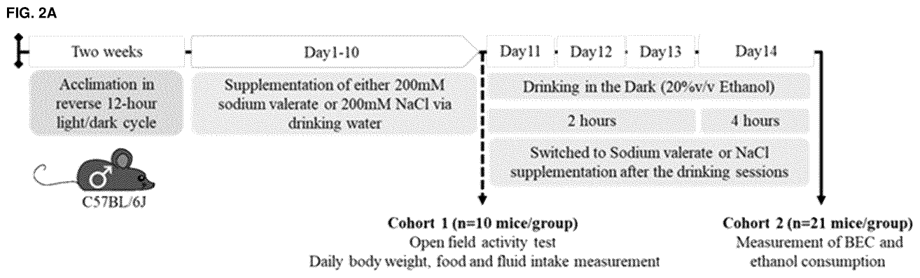

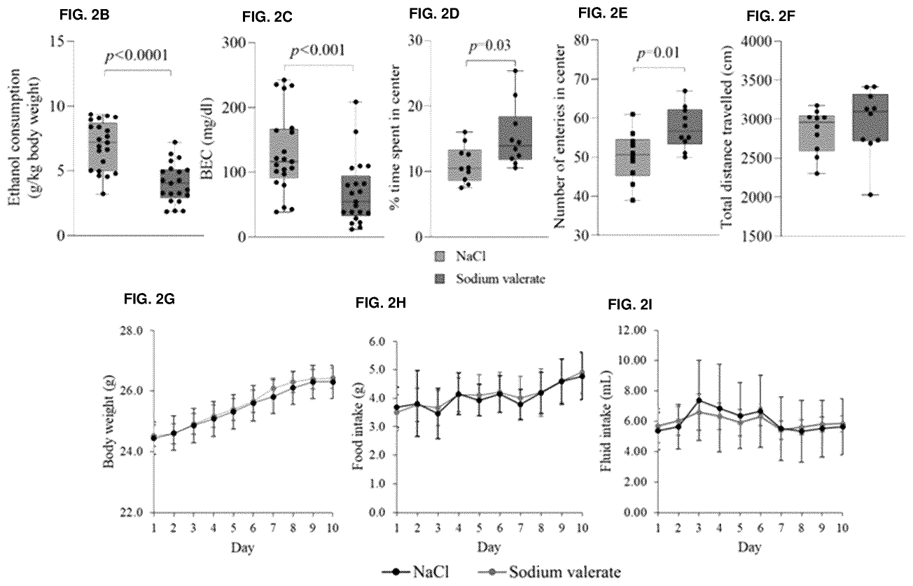

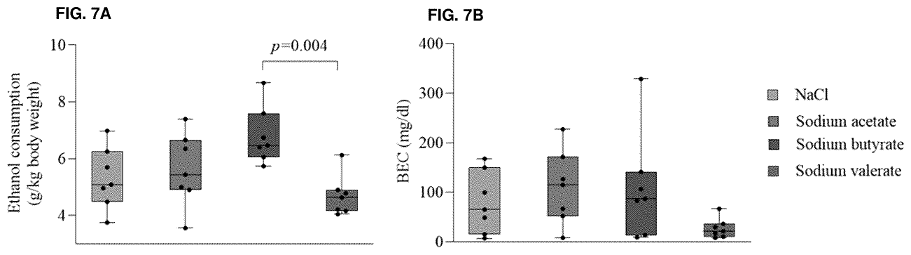

Next, the effect of individual SCFAs (sodium butyrate, sodium acetate, sodium propionate, sodium valerate, vehicle control (NaCl) and no treatment control) supplementation (daily for 10 days in drinking water) was investigated on ethanol consumption in mice (n=7 mice/group) via the DID paradigm. A significant difference (p=0.004, f=5.04 and df=24) was observed between sodium valerate and sodium butyrate supplemented mice (FIGS. 7A-7B). The sodium valerate supplementation study was repeated in a larger cohort of male mice (n=21 mice/group) (FIG. 2A). The effect of sodium valerate supplementation on reducing ethanol consumption (p<0.0001, t=4.09 and df=54; FIG. 2B) and BEC (p<0.001, t=3.53 and df=40; FIG. 2C) was significant. Sodium valerate supplementation led to a 40% reduction in ethanol consumption, with a median of 4.03 g/kg body weight compared to 7.16 g/kg consumed body weight in the NaCl supplemented mice. Additionally, BEC was lowered by 53% in sodium valerate-supplemented mice, with a median of 54.3 mg/dl compared to a median of 116.3 mg/dl in NaCl supplemented control mice.

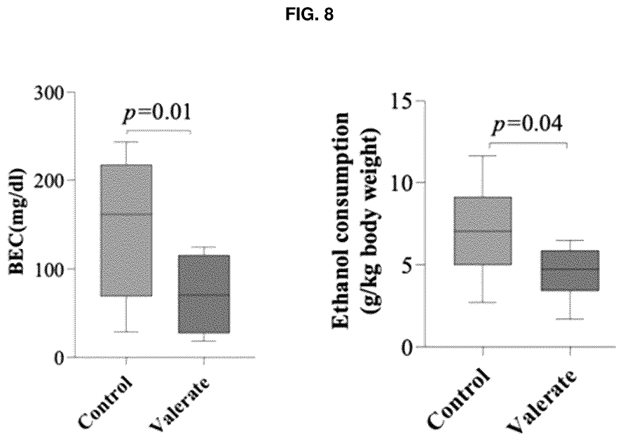

Inventors also tested the effect of sodium valerate on drinking and blood ethanol levels using female mice (n=7/group) using the same DID protocol. Sodium valerate supplementation significantly reduced alcohol binge drinking and blood ethanol levels in female mice (FIG. 8).

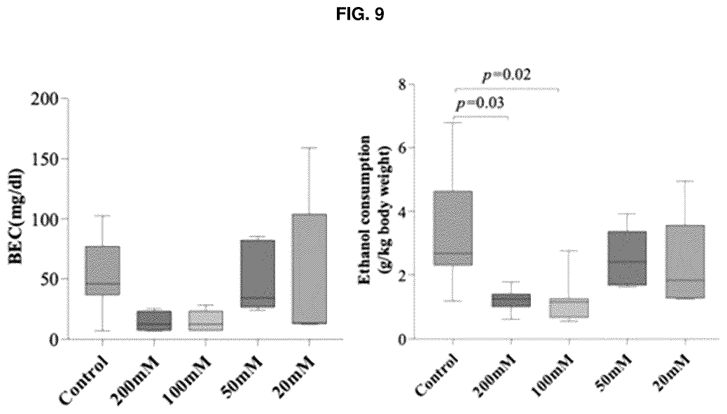

To determine dosage dependent effect of sodium valerate and also test whether short-term administration have any effect on drinking and blood alcohol levels, sodium valerate was administered at dosage ranging from 20-50 mM by drinking water 30 min before 2 h alcohol exposure at day 1 of the 4-day DID regimen and treatment continued until day 4. It was found that 100 mM and 200 mM sodium valerate treatment 30 min before alcohol exposure significantly decreased alcohol intake. (FIG. 9)

The effects of sodium valerate and NaCl was examined on anxiety-like behavior (n=10 mice/group) after 10 days of supplementation by open-field activity test. As shown in FIGS. 2D-2E, sodium valerate supplementation produced a significant increase in percentage of time spent in center are [mean±SD=15.29±4.82; p=0.03, t=2.36 and df=18] and number of entries in center [mean±SD=57.50±5.48; p=0.01, t=2.73 and df=18] during a 10 min exploration period. However, it did not lead to a significant change in the distance travelled (FIG. 2F). Overall, supplementation of sodium valerate reduces anxiety-like or approach-avoidance behavior in mice. Although the mice gained weight throughout the study, the average weight did not differ significantly (n=10 mice/group) between the sodium valerate and NaCl groups (FIG. 2G). In addition, no statistical difference was observed (n=14 mice/group) in food consumption or intake of sodium valerate and NaCl among the respective groups (FIGS. 2H and 2I).

### Enhanced GABA Levels Following Sodium Valerate Supplementation

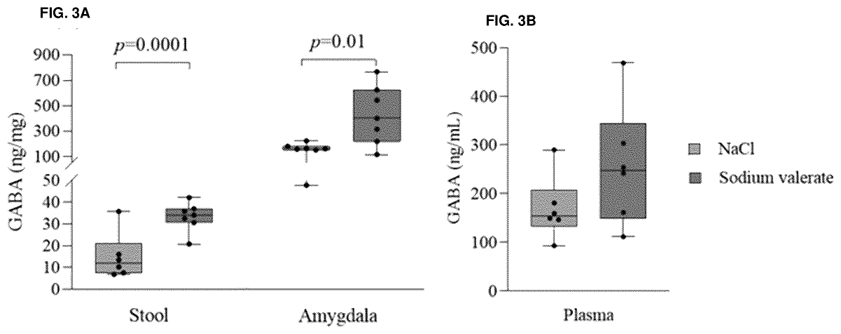

The levels of GABA in the amygdala were measured next, stool, and blood of the mice that underwent the DID paradigm, comparing those supplemented with sodium valerate to those supplemented with NaCl. The findings revealed that sodium valerate supplementation led to elevated GABA levels in stool (p=0.0001, t=5.71, df=11) and the amygdala (p=0.01, t=2.78, df=12), compared to the control group supplemented with NaCl (FIG. 3A). A similar trend was also evident in plasma (FIG. 3B) but was not significant. No detectable levels were observed when directly testing valeric acid with the ELISA assay.

### Sodium Valerate Supplementation Increases Histone Acetylation in the Amygdala

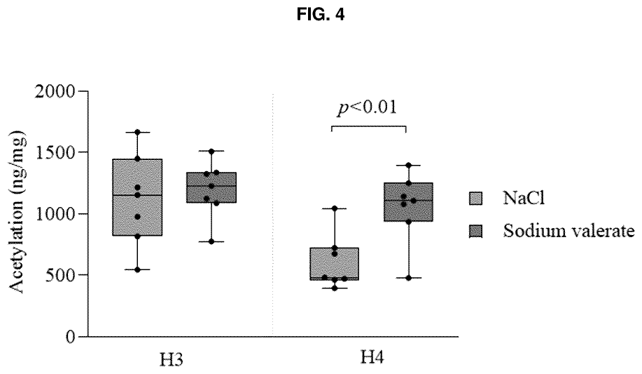

The total acetylation levels of histone H3 and H4 were quantified in the amygdala of mice subjected to the DID paradigm following supplementation with sodium valerate or NaCl (n=7 mice/group). It was found H4 acetylation significantly increased in mice supplemented with sodium valerate compared to NaCl control (p<0.01, t=3.20, df=12), while the observed increase in H3 acetylation with sodium valerate was not statistically significant in the amygdala of these mice (FIG. 4).

Amygdala Transcriptome Analysis in Mice Supplemented with Sodium Valerate

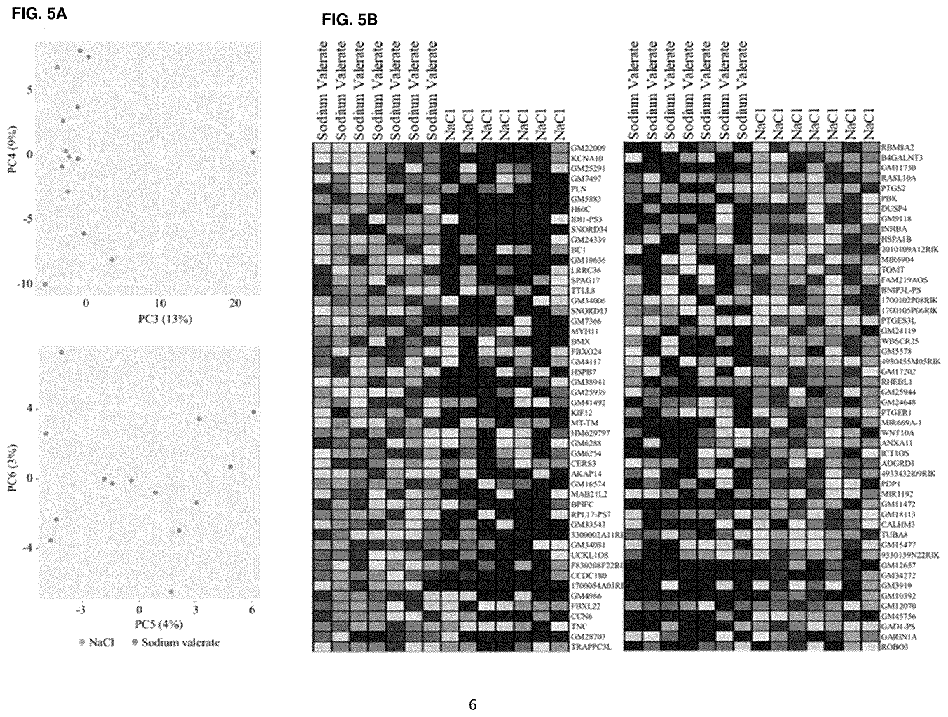

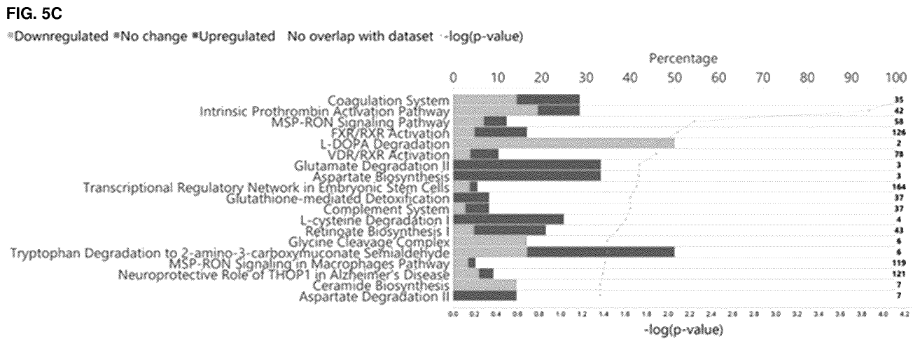

To gain a molecular understanding of the effects of sodium valerate supplementation on brain function, bulk RNA sequencing (RNA-seq) analysis was performed on amygdala tissue of mice that underwent the DID paradigm after sodium valerate or NaCl (n=7 mice/group) supplementation. Principal component analysis (PCA) showed no clear distinction between sodium valerate supplemented mice and NaCl supplemented mice at PC1 and PC2 components, but a clear separation in PC3 to PC6 (FIG. 5A), indicating sodium valerate supplementation has a marked influence on gene expression in the amygdala. DESeq2 identified a total of 301 DEG at the adjusted p value <0.25, 126 genes at the adjusted p value <0.10, and 75 genes at the adjusted p value <0.05. Among the 75 significant DEG, 47 genes showed upregulation, while 28 displayed downregulation. The heatmap displays the top 50 genes in the sodium valerate versus NaCl control (FIG. 5B). Among these genes, the top five protein coding genes with the highest expression in sodium valerate-supplemented mice are Kcna10 (potassium voltage-gated channel, shaker-related subfamily, member 10), Pln (phospholamban), H60C (histocompatibility antigen 60c), Idi1 (isopentenyl-diphosphate delta isomerase, pseudogene 3) and Snord34 (small nucleolar RNA, C/D box 34). The top five most strongly down-regulated genes in the sodium valerate group are Rbm8a2 (RNA binding motif protein 8A2), B4galnt3 (beta-1,4-N-acetyl-galactosaminyl transferase 3), Rasl10a (RAS Like Family 10 Member A), Pigs2 (prostaglandin-endoperoxide synthase 2) and Pbk (PDZ binding kinase). PTGS2 is a prostanoids producer that responds to inflammation. Its downregulation in the sodium valerate supplemented group (log₂Fold change: −0.65; adjusted p=0.0002) indicates an anti-inflammatory effect of valerate. Mitogen-activated protein kinases (MAPKs), a group of protein kinases that regulate critical inflammatory processes are also downregulated at the RNA level in the sodium valerate supplemented group. As SCFAs may function in the brain through G protein-coupled receptors (GPCRs), gene expression levels of 85 GPRs were examined in the RNAseq data. It was found that GPR56 and GPR158 are highly expressed among all these GPCRs. Gpr56, whose expression has been associated with antidepressant response, was upregulated in sodium valerate supplemented mice (log₂Fold change: 0.28; unadjusted p=0<0.01). Conversely, GPR158 is associated with depression after chronic stress, and is downregulated in the sodium valerate supplemented group (log₂Fold change: −0.26; unadjusted p<0.01). Notably, it was found that six mitochondrial genes encoding subunits of the enzyme NADH dehydrogenase (Complex I) were consistently upregulated in the sodium valerate supplemented group. IPA was performed to identify changes to the molecular signaling pathways and to comparatively assess the sodium valerate effects in the amygdala. The regulated pathways predicted in sodium valerate supplemented mice are associated with downregulation of L-dopa degradation, upregulation of aspartate biosynthesis, glutamate degradation, tryptophane degradation, the bidirectional regulation of pulmonary blood coagulation, retinoate biosynthesis, complement system, FXR/RXR activation, LXR/RXR activation, as well as the upregulation of L-cysteine degradation and the downregulation of glycine cleavage complex ceramide biosynthesis, etc. (FIG. 5C). Thus, sodium valerate treatment induces significant changes in gene expression spanning various signaling processes including neuroinflammation, neurotransmission, mitochondria regulation, and GPCR signaling.

### Sodium Valerate Supplementation Shifts the Gut Microbiota Community and Gut-Brain Modules

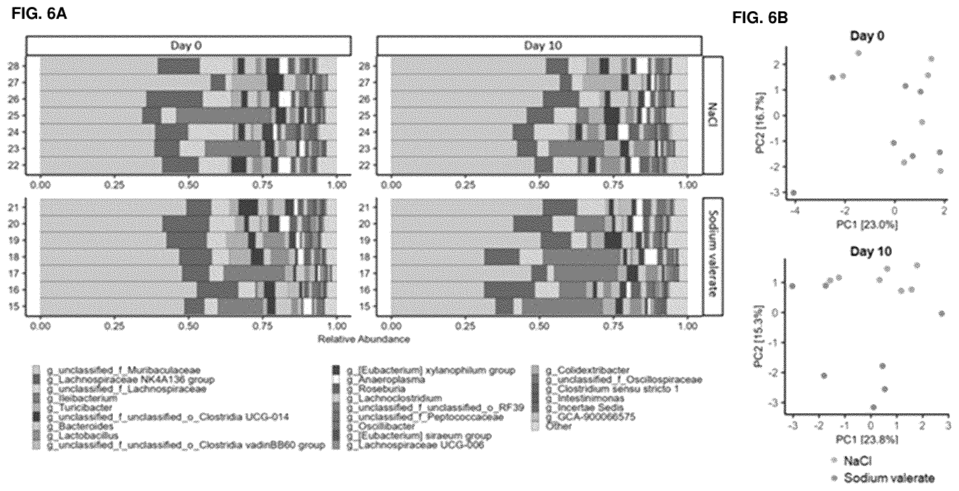

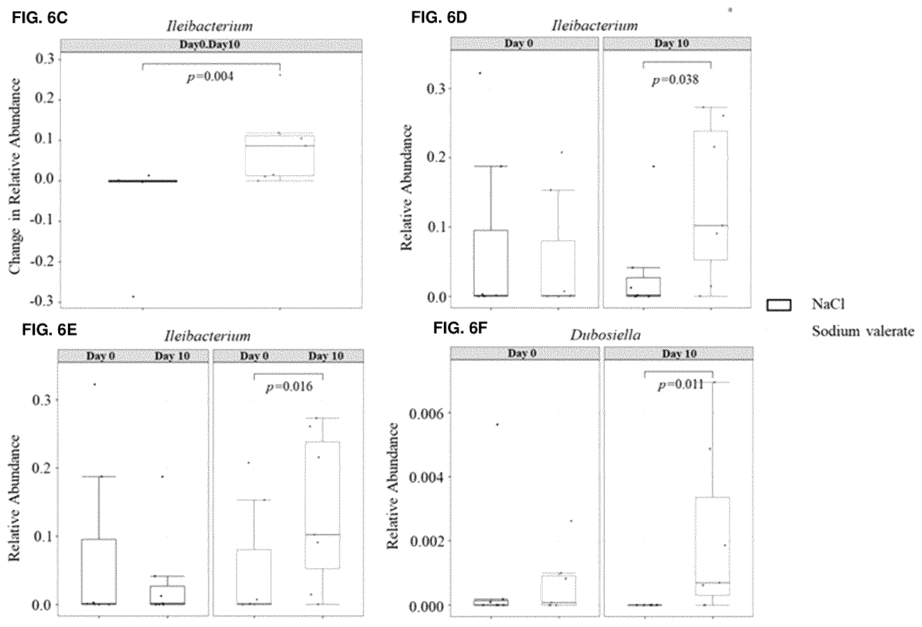

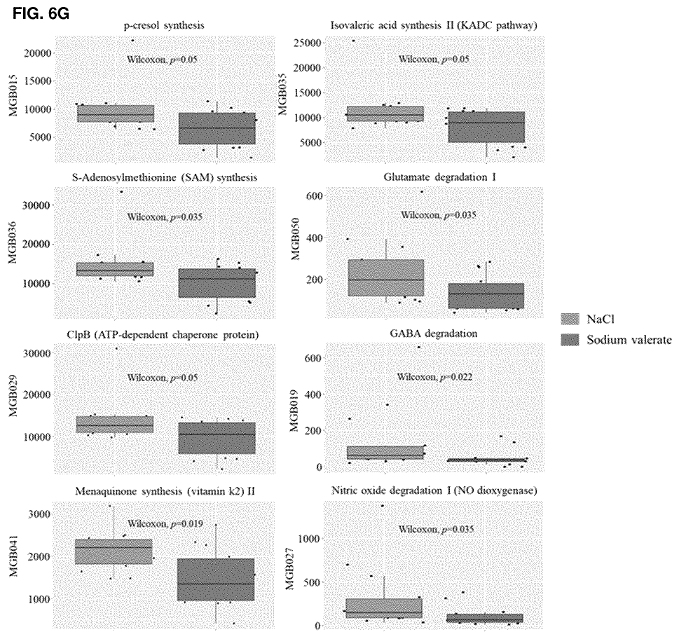

16S rRNA gene sequencing of stool samples collected before and after sodium valerate treatment (n=7 mice/group) was performed. The most abundant genera were unclassified Muribaculaceae, Lachnospiraceae NK4A136 group, and unclassified Lachnospiraceae (FIG. 6A). Permutational multivariate analysis of variance (PERMANOVA) tests were performed on CLR-transformed data to test for beta-diversity differences between sodium valerate and NaCl-supplemented groups (n=14 mice/group). Prior to any supplementation at day 0, no significant difference was observed between the groups. However, after 10 days of supplementation, the groups showed increased dissimilarity and exhibited distinct clustering patterns when visualized in a PCA plot, indicating a supplementation-related impact on the composition of the gut microbiome (FIG. 6B). Among all the genera analyzed, an increase in abundance was noted only in the Ileibacterium genera in sodium valerate supplemented mice (adjusted p=0.24, unadjusted p=0.004) (FIG. 6C). This finding was further confirmed via Wilcox tests on the relative abundance at day 10 between sodium valerate and NaCl mice (unadjusted p=0.038) (FIG. 6D), and the difference in abundance between day 0 and day 10 for the sodium valerate mice (unadjusted p=0.011), which supported an increase in Ileibacterium in sodium valerate supplemented mice (FIG. 6E). Further analysis with DESeq2 found that the genus Dubosiella was significantly (log₂Fold change=7.84, adjusted p=9.46E-5) more abundant in mice after 10 days of sodium valerate supplementation (FIG. 6F). This finding was confirmed with a Wilcox test (unadjusted p=0.011).

To determine neuroactive potential of the valerate-altered gut microbiota, neuroactive compound production and degradation process based on gut-brain module (GBM) analysis was inferred. Thirty-four GBMs were identified in the collected samples, 8 GBMs exhibited significant differences (p<0.05) between the sodium valerate and NaCl supplemented mice. Interestingly, all 8 GBMs showed a significant decrease in valerate treated mice (FIG. 6G), including p-Cresol synthesis, Isovaleric acid synthesis II, S-Adenosylmethionine synthesis, Glutamate degradation I, GABA degradation, Nitric oxide degradation I, ClpB (ATP-dependent chaperone protein), and Menaquinone synthesis (vitamin K2) II.

### Discussion

Inventor has discovered that a sodium salt of gut microbial metabolite valeric acid reduces binge-like alcohol consumption in mice. This effect is associated with an increase of GABA levels in the periphery and brain, modulation of brain epigenetics and transcriptomics, and an impact on the gut microbiome composition.

Previous research has demonstrated the involvement of gut microbiota in alcohol consumption, and the disruption of gut microbiota through antibiotics has been shown to increase ethanol consumption in mice. Inventor has confirmed that Abx treatment significantly increased voluntary ethanol consumption levels in a binge-like ethanol drinking paradigm in mice. However, the opposite effect was reported in wistar-derived high-drinker UChB rats [Ezquer F., et al., Innate gut microbiota predisposes to high alcohol consumption. Addiction biology 2021, 26 (4): e13018], it's worth noting that it utilized an ad libitum ethanol access paradigm leading to lower BEC. In addition, a different antibiotics regimen, and animals were also used in their study. Regardless, this work reveals an important relationship between the gut microbiota and ethanol consumption behavior and supports the use of microbial-targeted approaches to study gut-brain interactions in alcohol drinking behavior. In mouse model studied herein, the production of intestinal SCFAs, including acetate, butyrate, isobutyrate, propionate, valerate, and isovalerate, was significantly suppressed by the Abx treatment.

When provided various SCFAs to mice as supplements, no statistical changes were observed in alcohol intake when sodium acetate and butyrate were supplemented. Prior research suggests that acetate might encourage heavy drinking, providing a reward in the form of added energy from calories or by influencing adenosinergic adaptation mechanisms [Jiang L., et al., Increased brain uptake and oxidation of acetate in heavy drinkers. The Journal of clinical investigation 2013, 123 (4): 1605-1614]. Studies have shown that sodium butyrate does not influence alcohol self-administration in non-dependent rodents but may reduce drinking in alcohol-dependent or antibiotic treated rodents [Reyes R E, et al., Supplementation with sodium butyrate protects against antibiotic-induced increases in ethanol consumption behavior in mice. Alcohol 2022, 100:1-9; Simon-O'Brien E., et al., The histone deacetylase inhibitor sodium butyrate decreases excessive ethanol intake in dependent animals. Addiction biology 2015, 20 (4): 676-689]. Interestingly, when valeric acid was supplemented, a significant reduction in ethanol consumption and BEC was observed. According to reports, BEC in humans ranging from 50-70 mg/dl may result in mild impairment in motor skills. In this study, mice treated with sodium valerate exhibited BEC of approximately 54.3 mg/dl, which is relatively low.

Valeric acid also presents naturally in various plant sources, including Valeriana wallichii and Valeriana officinalis etc. One study has shown that Valeriana wallichii extract reduces chronic ethanol intake in animal models. However, Valeriana wallichii extract contains a variety of active constituents. The exact compound that is responsible for reduced ethanol intake has not been studied. In this study, sodium valerate supplementation did not affect body weight, food intake, or fluid drinking. This shows that the observed reduction in alcohol consumption was not due to changes in fluid or weight regulation.

The molecular mechanisms that underlie alcohol drinking behaviors are intricate and multifaceted. Anxiety can promote alcohol drinking behaviors in both humans and animals, and excessive drinking increase anxiety-like behavior. This study indicates that sodium valerate supplementation has anxiolytic effects in mice. Interestingly, Valeriana wallichii and Valeriana officinalis, plant reservoirs of valeric acid and other compounds, have been used as supplements to address insomnia and anxiety due to their sedative attributes. GABA may play a role in reducing depression and anxiety linked to alcohol dependence, as lower GABA levels are associated with these conditions. In this study, sodium valerate supplement led to increased GABA levels in stool and the amygdala.

Further, increased levels of GABA detected in stool samples from mice supplemented with sodium valerate indicate that the gut microbiome may be involved in GABA regulation. Previous studies have identified a variety of GABA-producers and degraders in the gut microbiome. Indeed, the gut-brain module analysis revealed a decrease in GABA degradation by the gut microbiome in sodium valerate supplemented mice. It will be interesting to examine the ability of GABA modulation by Ileibacterium and Dubosiella, two bacterial genera that are significantly increased during sodium valerate supplementation. However, it is also possible that changes in Ileibacterium and Dubosiella abundance are responses to administered sodium valerate or increased GABA. A report indicated that the metabolic disorder induced by chronic alcohol consumption caused a decrease in the relative abundance of Ileibacterium-[Liu Y., et al., Gut Microbiome and Metabolome Response of Pu-erh Tea on Metabolism Disorder Induced by Chronic Alcohol Consumption. Journal of agricultural and food chemistry 2020, 68 (24): 6615-6627]. Under physiological conditions, it has been widely believed that GABA does not cross blood brain barrier, whereas SCFAs have the ability of brain penetration. The impact of gut-derived GABA on brain function and drinking behavior, therefore, warrants further investigation. Without wishing to be bound by this theory it is hypothesized that valeric acid can directly cross blood brain barrier and regulates GABA levels in the brain or acts indirectly through gut-brain axis. These observations suggest a connection between valeric acid supplementation, heightened GABA levels, and a reduction in alcohol consumption.

There is consistent evidence that acute and chronic alcohol exposure modulate histone acetylation in the amygdaloid circuitry, leading to alcohol tolerance and dependence. This study revealed increased acetylation of histone H4 in the amygdala of sodium valerate-supplemented mice. Previous findings confirm that intermittent alcohol exposure decreased histone acetylation in the amygdala, which may be related to the ethanol-induced increase in histone deacetylase (HDAC). Similarly, administration of HDAC inhibitors like sodium butyrate increase histone acetylation and suppress anxiety or depression-like behaviors in mice. Results disclosed herein suggest that HDAC inhibitors such as sodium valerate may be able to reverse the effects of ethanol via HDAC-induced epigenetic changes in the amygdala, consequently resulting in reduced ethanol consumption.

Increased histone acetylation leads to a more open structure of chromosomes, thus promoting gene transcription. Disclosed data indicates that another potential mechanism by which valeric acid attenuates alcohol drinking is through its effects on transcriptional regulation in the brain. A downregulation of inflammatory molecules such as Ptgs2 and MAPK was identified in valerate-treated mice. The immune modulatory effect of valerate has been shown in experimental mouse models of colitis and multiple sclerosis, mediated by suppressing Th 17 cells and the enhancement of IL-10 production. Numerous studies have demonstrated that SCFAs modulate transcription of a wide range of genes associated with behaviors. SCFAs are known to regulate GPCRs such as GPR41, GPR43, and GPR109A, all of which are critical in regulating neuroinflammation, depression, and anxiety-like behaviors. The bulk RNAseq analysis disclosed herein showed upregulation of GPR56 and downregulation of GPR158 in the amygdala region of the brain in valerate treated mice. GPR158 is a novel regulator of stress-responsive behaviors and is highly upregulated in people with major depression disorder. By contrast, GPR 56 activation has an antidepressant effect. Without wishing to be bound by this theory it is hypothesized that Valerate acid may regulate two GPRs of opposing effects to control anxiety or depression behavior, thus indirectly influencing moderating drinking behavior.

Sodium valerate supplementation shows promise as a novel intervention to reduce alcohol consumption. This effect potentially acts through the modulation of multiple molecular targets associated with the pathogenesis of excessive alcohol use. This study contributes to the growing understanding of the gut-brain axis and provides insights into potential therapeutic strategies for excessive alcohol consumption and related anxiety.

### Example 3. Effect of Effect of Valerate on Repeated DID Sessions in Mice

In this study, the impact of sodium valerate treatment on ethanol intake and blood ethanol concentration (BEC) was assessed using a six-week Drinking in the Dark (DID) protocol in male mice (C57BL/6J). All mice (n=30) were single-housed and had ad libitum access to food and their respective drinking water. Water bottles were removed 30 minutes prior to and throughout each DID session. During the two-week baseline period, all mice underwent a standardized DID paradigm consisting of a 4-day cycle each week: 2-hour access to 20% ethanol on days 1-3, followed by a 4-hour session on day 4. Starting in week three, mice were randomly assigned to one of three groups (10 mice per group): (1) Valerate-i.p., which received intraperitoneal injections of sodium valerate (200 mg/kg body weight) 30 minutes before each DID session; (2) Valerate-drinking water, which received 200 mM sodium valerate in drinking water for 30 minutes before to each DID session; and (3) Control, which received no valerate treatment. Ethanol consumption was evaluated on day 4 of each week (Wecks 1-6), and BEC was measured on day 4 of weeks 2, 4, and 6.

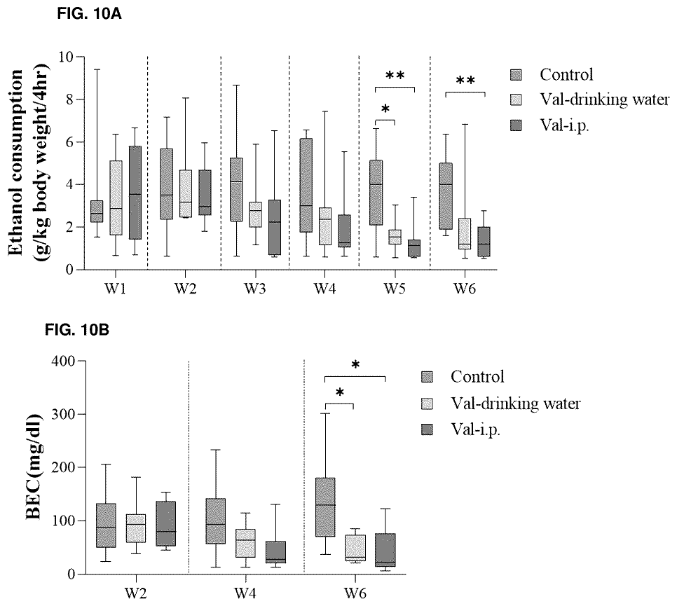

The ethanol consumption was significantly reduced in week 5 for both treatment groups relative to control (p<0.01 for Valerate-i.p.; p<0.05 for Valerate-drinking water; FIG. 10A). Interestingly, in week 6, only the Valcrate-i.p. group showed a trend toward reduced ethanol consumption (p<0.1; FIG. 10A). In week 6, both valerate-treated groups exhibited significant reductions in BEC compared to the control group (p<0.01 for both groups; two-way ANOVA with Tukey's post hoc correction; FIG. 10B). No significant differences were observed between the two valerate-treated groups. Additionally, no significant effects were noticed during the initial weeks of treatment, indicating that the impact of valerate accumulates with repeated treatment. These results show that sodium valerate may be a potential intervention for reducing excessive alcohol consumption over time.

### Example 4. Effect of Valerate on Intermittent-Access 20% Ethanol 2-Bottle Choice Drinking in Mice

Twenty male C57BL/6J mice were given access to 20% ethanol for three 24-hour sessions per week. On the first Monday, Wednesday, and Friday, mice were offered increasing concentrations of ethanol (3%, 6%, and 10% w/v, respectively) in one bottle, with water in a second bottle, to acclimate them to ethanol consumption. After this acclimation, mice received one bottle of 20% ethanol and one bottle of water every Monday, Wednesday, and Friday for 24 hours. To minimize side bias, the position of the bottles in the cage was alternated between sessions.

For the first 8 weeks, mice received intraperitoneal (i.p.) injections of phosphate-buffered saline (PBS) 30 minutes prior to each ethanol session to establish stable ethanol consumption (g/kg) and ethanol preference (ethanol intake as a proportion of total fluid intake). In the following 4 weeks (12 sessions), mice were randomly assigned to receive i.p. injections of either sodium valerate or sodium chloride (NaCl) at a dose of 600 mg/kg, 30 minutes before each ethanol session (n=10 per group). Ethanol and water consumption, as well as ethanol preference, were measured throughout the study to assess the effects of sodium valerate on ethanol drinking behavior compared to the NaCl control. After each 24-hour access period, the ethanol and water bottles were weighed, and mouse body weights were recorded to calculate ethanol intake (g/kg/24 hours) and water intake (mL/kg/24 hours). Ethanol preference was calculated as the ratio of ethanol intake to total fluid intake.

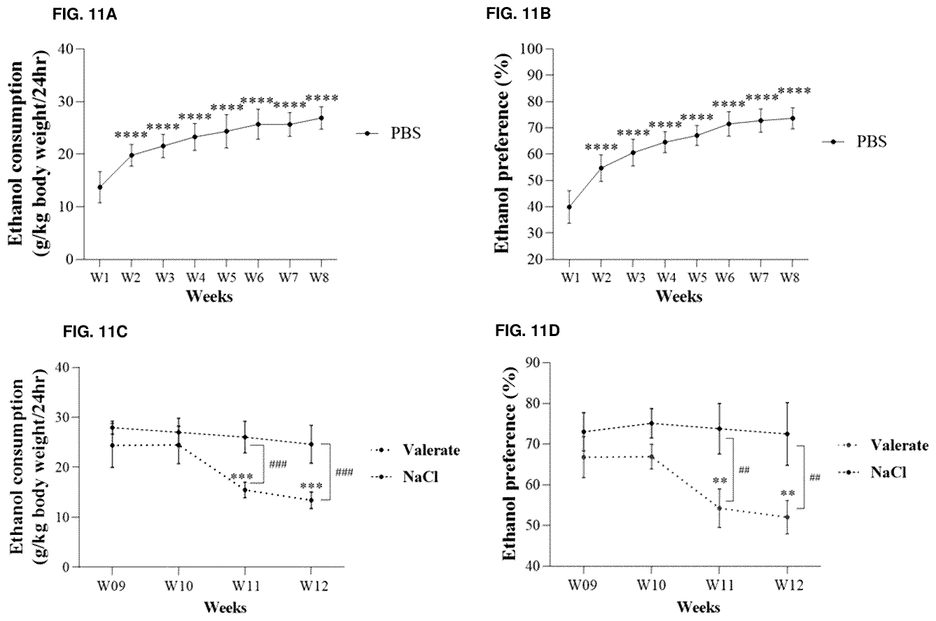

The intermittent-access 20% ethanol two-bottle-choice drinking paradigm resulted in a significant escalation of ethanol consumption and preference in the mice (n=20). ANOVA and post hoc analyses revealed a significant increase in ethanol consumption from week 1 to week 8 [from 13.72±2.93 to 26.88±2.10 g/kg/24 hours; p<0.0001; FIG. 11A], accompanied by a significant rise in ethanol preference [from 39.88±6.08% to 73.65±3.93%; p<0.0001; FIG. 11B]. Ethanol intake and preference stabilized after the sixth week through the eighth week.

During the subsequent 4-week treatment phase (12 sessions), beginning in week 11, sodium valerate-treated mice demonstrated a significant within-group reduction in both ethanol consumption and preference. Ethanol consumption decreased from 24.38±4.19 to 15.47±1.49 g/kg/24 hours (p<0.001; FIG. 11C), while ethanol preference declined from 66.81±4.8% to 54.26±4.5% (p<0.01; FIG. 11D). This reduction persisted into week 12, with further decreases in ethanol intake to 13.42±1.60 g/kg/24 hours (p<0.001; FIG. 11C) and preference to 52.06±3.92% (p<0.01; FIG. 11D). Compared to the NaCl control group, sodium valerate-treated mice showed significantly lower ethanol consumption (p<0.001 for both weeks) and preference (p<0.01 for both weeks). These findings demonstrate that sodium valerate significantly attenuates ethanol consumption and preference in mice.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

### INCORPORATION BY REFERENCE

All U.S. and PCT patent publications and U.S. patents mentioned herein are hereby incorporated by reference in their entirety as if each individual patent publication or patent was specifically and individually indicated to be incorporated by reference. In case of conflict, the present application, including any definitions herein, will control.

### OTHER EMBODIMENTS

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

### REFERENCE

1. Zhou Y, et al. The upper-airway microbiota and loss of asthma control among asthmatic children. Nat Commun. 2019; 10 (1): 5714. doi: 10.1038/s41467-019-13698-x
2. Jackson D J, et al. Quintupling Inhaled Glucocorticoids to Prevent Childhood Asthma Exacerbations. N Engl J Med. 2018; 378 (10): 891-901. doi: 10.1056/NEJMoa1710988
3. Qi Y., et al., Sodium acetate regulates milk fat synthesis through the activation of GPR41/GPR43 signaling pathway. Frontiers in nutrition 2023, 10:1098715
4. Daien C I, et al., Gut-derived acetate promotes B10 cells with anti-inflammatory effects. JCI insight 2021, 6 (7)
5. Ezquer F., et al., Innate gut microbiota predisposes to high alcohol consumption. Addiction biology 2021, 26 (4): e13018
6. Jiang L., et al., Increased brain uptake and oxidation of acetate in heavy drinkers. The Journal of clinical investigation 2013, 123 (4): 1605-1614
7. Reyes R E, et al., Supplementation with sodium butyrate protects against antibiotic-induced increases in ethanol consumption behavior in mice. Alcohol 2022, 100:1-9;
8. Simon-O'Brien E., et al., The histone deacetylase inhibitor sodium butyrate decreases excessive ethanol intake in dependent animals. Addiction biology 2015, 20 (4): 676-689
9. Liu Y., et al., Gut Microbiome and Metabolome Response of Pu-erh Tea on Metabolism Disorder Induced by Chronic Alcohol Consumption. Journal of agricultural and food chemistry 2020, 68 (24): 6615-6627

1. A method of treating or preventing alcohol use disorder or alcohol risk consumption in a subject in need thereof, comprising administering to the subject an effective amount of valeric acid, or a pharmaceutically acceptable salt thereof.

2. A method of reducing alcohol consumption in a subject in need thereof, comprising administering to the subject an effective amount of valeric acid or a pharmaceutically acceptable salt thereof.

3. The method of claim 1, wherein the pharmaceutically acceptable salt is sodium valerate.

4. The method of claim 1, further comprising administering one or more microbiome bacteria that improve valeric acid concentration in the subject, optionally wherein the one or more microbiome bacteria comprise Lactobacillus and/or Bifidobacteria.

5. (canceled)

6. The method of claim 1, wherein
(i) the amount and/or frequency of alcohol consumption of the subject is reduced;
(ii) the amount of alcohol consumption of the subject is reduced at least 10%, 30%, 50%, 70%, 90%, or 95%; and/or
(iii) the frequency of alcohol consumption of the subject is reduced to no more than once a week, once every two weeks, or once a month.

7.-8. (canceled)

9. The method of claim 1, wherein
(i) the expression level of one or more genes selected from the group consisting of GPR56, KCNA10, PLN, H60C, IDI1, and SNORD34 is increased; and/or wherein the expression level of one or more genes selected from the group consisting of GPR56, KCNA10, PLN, H60C, IDI1, and SNORD34 is increased by at least 1.2, 1.5, 2, 2.5, 3, 3.5, 4, 5, 6, 7, 8, 9, or 10 times; and/or
(ii) the expression level of one or more genes selected from the group consisting of GPR158, RBM8A2, B4GALNT3, RASL10A, PTGS2, and PBK is decreased; and/or wherein the expression level of one or more genes selected from the group consisting of GPR158, RBM8A2, B4GALNT3, RASL10A, PTGS2, and PBK is decreased by at least 10%, 30%, 50%, 70%, 90%, or 95%.

10.-12. (canceled)

13. The method of claim 1, wherein
(i) the level of one or more inflammatory molecules in the subject is decreased; wherein the level of one or more inflammatory molecules in the subject is decreased by at least 10%, 30%, 50%, 70%, 90%, or 95%; and/or wherein the one or more inflammatory molecules comprise prostaglandin-endoperoxide synthase 2 (PTGS2) and/or a mitogen-activated protein kinase (MAPK);
(ii) the level of one or more bacteria of gut microbiome in the subject is increased; wherein the level of one or more bacteria of gut microbiome in the subject is increased by at least 1.2, 1.5, 2, 2.5, 3, 3.5, 4, 5, 6, 7, 8, 9, or 10 times; and/or wherein the one or more bacteria comprise Ileibacterium and/or Dubosiella; and/or
(iii) the level of gamma-aminobutyric acid (GABA) in the subject is increased; wherein the level of GABA in the subject is increased at least by 1.2, 1.5, 2, 2.5, 3, 3.5, 4, 5, 6, 7, 8, 9, or 10 times; and/or wherein the level of GAPA is increased in stool and/or amygdala of the subject.

14.-21. (canceled)

22. The method of claim 1, the level of anxiety of the subject is reduced.

23. The method of claim 1, wherein the valeric acid, or a pharmaceutically acceptable salt thereof, is administered orally.

24. The method of claim 1, which does not comprise administering carnitine or a derivative thereof to the subject.

25. A method of increasing the level of one or more bacteria of gut microbiome in a subject, comprising administering to the subject an effective amount of valeric acid, or a pharmaceutically acceptable salt thereof, wherein the one or more bacteria comprise Ileibacterium and/or Dubosiella.

26. The method of claim 25, wherein the subject has alcohol use disorder.

27. The method of claim 26, wherein
(i) the amount and/or frequency of alcohol consumption of the subject is reduced;
(ii) the amount of alcohol consumption of the subject is reduced at least 10%, 30%, 50%, 70%, 90%, or 95%; and/or
(iii) the frequency of alcohol consumption of the subject is reduced to no more than once a week, once every two weeks, or once a month.

28. (canceled)

29. (canceled)

30. The method of claim 26, wherein the pharmaceutically acceptable salt is sodium valerate.

31. The method of claim 26, wherein
(i) the expression level of one or more genes selected from the group consisting of GPR56, KCNA10, PLN, H60C, IDI1, and SNORD34 is increased; and/or wherein the expression level of one or more genes selected from the group consisting of GPR56, KCNA10, PLN, H60C, IDI1, and SNORD34 is increased by at least 1.2, 1.5, 2, 2.5, 3, 3.5, 4, 5, 6, 7, 8, 9, or 10 times; and/or
(ii) the expression level of one or more genes selected from the group consisting of GPR158, RBM8A2, B4GALNT3, RASL10A, PTGS2, and PBK is decreased; and/or wherein the expression level of one or more genes selected from the group consisting of GPR158, RBM8A2, B4GALNT3, RASL10A, PTGS2, and PBK is decreased by at least 10%, 30%, 50%, 70%, 90%, or 95%.

32.-34. (canceled)

35. The method of claim 26, wherein
(i) the level of one or more inflammatory molecules in the subject is decreased; wherein the level of one or more inflammatory molecules in the subject is decreased by at least 10%, 30%, 50%, 70%, 90%, or 95%; and/or wherein the one or more inflammatory molecules comprise prostaglandin-endoperoxide synthase 2 (PTGS2) and/or a mitogen-activated protein kinase (MAPK); and/or
(ii) the level of gamma-aminobutyric acid (GABA) in the subject is increased; wherein the level of GABA in the subject is increased at least by 1.2, 1.5, 2, 2.5, 3, 3.5, 4, 5, 6, 7, 8, 9, or 10 times; and/or wherein the level of GAPA is increased in stool and/or amygdala of the subject.

36.-40. (canceled)

41. The method of claim 26, the level of anxiety of the subject is reduced.

42. The method of claim 26, wherein valeric acid, or a pharmaceutically acceptable salt thereof, is administered orally.

43. The method of claim 26, which does not comprises administering carnitine or a derivative thereof to the subject.
